(19) United States
(12) Patent Application Publication
(10) Pub. No.: US 2018/0132827 A1
(43) Pub. Date: May 17, 2018

(54) ULTRASONIC UNIT, ULTRASONIC APPARATUS, AND CONTROL METHOD FOR ULTRASONIC UNIT

(71) Applicant: Seiko Epson Corporation, Tokyo (JP)

(72) Inventors: Daisuke NAKANISHI, Matsumoto (JP); Kanechika KIYOSE, Matsumoto (JP)

(21) Appl. No.: 15/807,959

(22) Filed: Nov. 9, 2017

(30) Foreign Application Priority Data
Nov. 11, 2016 (JP) 2016-220472

(51) Int. Cl. A61B 8/00; A61B 8/15

(52) U.S. Cl. CPC A61B 8/4494; A61B 8/54; A61B 8/15

(57) ABSTRACT

An ultrasonic unit includes a first and a second ultrasonic wave transmission and reception portions that transmit ultrasonic waves to a subject and receive ultrasonic waves reflected from the subject, a first sound guide portion through which ultrasonic waves transmitted and received by a first ultrasonic wave transmission and reception portion pass, and a second sound guide portion through which ultrasonic waves transmitted and received by a second ultrasonic wave transmission and reception portion pass, in which a distance between the first sound guide portion and the second sound guide portion is gradually reduced toward the subject.

### BACKGROUND

### 1. Technical Field

The present invention relates to an ultrasonic unit, an ultrasonic apparatus, and a control method for the ultrasonic unit.

### 2. Related Art

In the related art, there is an ultrasonic probe or an ultrasonic apparatus which outputs an ultrasonic wave transmitted from an ultrasonic vibrator to a subject (for example, a living body) (for example, refer to JP-A-2011-35916).

JP-A-2011-35916 discloses an ultrasonic probe in which an ultrasonic medium fills between each ultrasonic vibrator and an acoustic lens, and an ultrasonic wave transmitted from the ultrasonic vibrator is emitted toward a subject from the acoustic lens. JP-A-2011-35916 also discloses a configuration in which an elastic body is provided in each ultrasonic vibrator, and an ultrasonic wave emission surface of the elastic body is an inclined surface. In this case, an ultrasonic wave is sent at an angle corresponding to the inclined surface.

Meanwhile, in the ultrasonic probe of the related art as disclosed in JP-A-2011-35916, a plurality of ultrasonic vibrators are provided on a single array substrate. However, in a case where transmission and reception of ultrasonic waves are necessary for a large area, in the configuration as disclosed in JP-A-2011-35916, it is necessary to increase an area of the array substrate, and thus an apparatus unit price tends to increase.

Therefore, transmission and reception of ultrasonic waves may be performed for a larger area by using a plurality of array substrates each having the same size as that of the array substrate of the related art. However, in this case, a region in which transmission and reception of ultrasonic waves are not performed is formed between the array substrates, and thus an ultrasonic wave emitted from the ultrasonic probe is not uniform. Also in a case where an ultrasonic wave is received, ultrasonic waves which are incident from a predetermined region include an ultrasonic wave forwarded to the array substrate and an ultrasonic wave forwarded to the region between the array substrates, and thus measurement accuracy is reduced. Consequently, the accuracy of transmission and reception of ultrasonic waves is reduced. For example, in a case where an internal tomographic image of a subject is formed on the basis of transmission and reception results of ultrasonic waves, there is a problem in that a highly accurate internal tomographic image cannot be formed due to a defect such as the presence of a region which is not reflected in a partial image.

### SUMMARY

An advantage of some aspects of the invention is to provide an ultrasonic unit, an ultrasonic apparatus and a control method for the ultrasonic unit capable of increasing the accuracy of transmission and reception of ultrasonic waves, and application examples and embodiments capable of achieving the advantage will be explained below.

An ultrasonic unit according to this application example includes a plurality of ultrasonic wave transmission and reception portions that transmit ultrasonic waves to a subject and receive ultrasonic waves reflected from the subject; a first sound guide portion through which ultrasonic waves transmitted and received by a first ultrasonic wave transmission and reception portion forming the plurality of ultrasonic wave transmission and reception portions pass; and a second sound guide portion through which ultrasonic waves transmitted and received by a second ultrasonic wave transmission and reception portion forming the plurality of ultrasonic wave transmission and reception portions pass, in which a distance between the first sound guide portion and the second sound guide portion is gradually reduced toward the subject.

The plurality of ultrasonic wave transmission and reception portions in the application example may be provided on different substrates, and may be provided on a single substrate.

In the application example, the ultrasonic unit is provided with the first sound guide portion through which ultrasonic waves transmitted and received by the first ultrasonic wave transmission and reception portion pass and the second sound guide portion through which ultrasonic waves transmitted and received by the second ultrasonic wave transmission and reception portion pass. A distance between the first sound guide portion and the second sound guide portion is gradually reduced toward the subject.

Consequently, even in a case where the first ultrasonic wave transmission and reception portion and the second ultrasonic wave transmission and reception portion are separated from each other, ultrasonic waves transmitted from the first sound guide portion or the second sound guide portion in a direction directed toward the subject are transmitted from positions close to each other. In other words, in a configuration in which the first sound guide portion or the second sound guide portion is not provided, a region in which an ultrasonic wave is not transmitted is present between an ultrasonic wave transmitted from the first ultrasonic wave transmission and reception portion toward the subject and an ultrasonic wave transmitted from the second ultrasonic wave transmission and reception portion toward the subject, and thus transmission density of the ultrasonic waves is nonuniform. In contrast, in the application example, since the first sound guide portion and the second sound guide portion are provided so that a distance therebetween is gradually reduced toward the subject, a distance between an ultrasonic wave transmitted from the first ultrasonic wave transmission and reception portion and an ultrasonic wave transmitted from the second ultrasonic wave transmission and reception portion is reduced, and thus an ultrasonic wave can be transmitted in uniform transmission density.

Also in a case where an ultrasonic wave is received, if the first sound guide portion or the second sound guide portion is not provided, an ultrasonic wave which is incident between the first ultrasonic wave transmission and reception portion and the second ultrasonic wave transmission and reception portion cannot be received. In contrast, in the application example, since a distance between the first sound guide portion and the second sound guide portion is gradually reduced toward the subject, ultrasonic waves from a subject can be caused to pass toward the first ultrasonic wave transmission and reception portion and the second ultrasonic wave transmission and reception portion, and thus a region in which an ultrasonic wave cannot be received is reduced.

As mentioned above, in the application example, ultrasonic waves transmitted and received are uniform, and thus it is possible to increase ultrasonic wave transmission and reception accuracy.

It is preferable that the ultrasonic unit according to the application example further includes an intermediate portion that has an acoustic impedance which is different from an acoustic impedance of each of the first sound guide portion and the second sound guide portion is provided between the first sound guide portion and the second sound guide portion.

In the application example with this configuration, the intermediate portion that has an acoustic impedance which is different from an acoustic impedance of each of the first sound guide portion and the second sound guide portion is provided between the first sound guide portion and the second sound guide portion. Consequently, since an ultrasonic wave is reflected by the intermediate portion, it is possible to prevent crosstalk in which an ultrasonic wave which has been incident to the first sound guide portion passes through the second sound guide portion side or an ultrasonic wave which has been incident to the second sound guide portion passes through the first sound guide portion side.

In the ultrasonic unit according to the application example, it is preferable that the intermediate portion includes an air layer.

In the application example with this configuration, the intermediate portion includes an air layer. Air has an acoustic impedance much lower than an acoustic impedance of an acoustic member such as silicone through which an ultrasonic wave propagates. Thus, if the intermediate portion is an air layer, or includes an air layer, it is possible to further reduce the above-described crosstalk.

In the ultrasonic unit according to the application example, it is preferable that facing side surfaces of the first sound guide portion and the second sound guide portion are inclined in intersecting directions toward the subject.

In the application example with this configuration, in a case where an ultrasonic wave is received, an ultrasonic wave which is incident to a position corresponding to a gap between the first ultrasonic wave transmission and reception portion and the second ultrasonic wave transmission and reception portion from a direction toward the subject is reflected at a first side surface or a second side surface, and thus an advancing direction thereof is changed to a direction along the first side surface or the second side surface. Thus, the ultrasonic wave whose advancing direction is changed is received by the first ultrasonic wave transmission and reception portion or the second ultrasonic wave transmission and reception portion, and therefore reception sensitivity can be improved.

In the ultrasonic unit according to the application example, it is preferable that a coupling surface of the first ultrasonic wave transmission and reception portion and the first sound guide portion and a coupling surface of the second ultrasonic wave transmission and reception portion and the second sound guide portion are located on different planes.

In the application example with this configuration, the first ultrasonic wave transmission and reception portion and the second ultrasonic wave transmission and reception portion are disposed along different planes and thus are not disposed on the same plane. For example, the first ultrasonic wave transmission and reception portion may be disposed on a first plane, and the second ultrasonic wave transmission and reception portion may be disposed on a second plane which is parallel to the first plane, and the second ultrasonic wave transmission and reception portion may be disposed on a second plane inclined with respect to a first plane. In this configuration, it is possible to improve a degree of freedom of arrangement of the first ultrasonic wave transmission and reception portion or the second ultrasonic wave transmission and reception portion.

It is preferable that the ultrasonic unit according to the application example further includes a first device including the first ultrasonic wave transmission and reception portion and the first sound guide portion; and a second device including the second ultrasonic wave transmission and reception portion and the second sound guide portion, and the first device and the second device are attachable to and detachable from each other.

In the application example with this configuration, the first device and the second device are attachable and detachable. Therefore, for example, in a case where ultrasonic measurement is desired to be performed on a region in a narrow range, the first device and the second device can be separated from each other, and ultrasonic measurement can be performed by using either one of the first device and the second device.

An ultrasonic apparatus according to this application example includes the ultrasonic unit described above; and a controller that controls the ultrasonic unit.

As described above, the ultrasonic unit can perform ultrasonic measurement with high accuracy by performing uniform ultrasonic wave transmission and reception processes on a subject. Thus, the ultrasonic apparatus using the ultrasonic unit can perform various processes (for example, a process of generating an internal tomographic image of a subject on the basis of an ultrasonic measurement result) based on a highly accurate ultrasonic measurement result, with high accuracy.

In the ultrasonic apparatus according to the application example, it is preferable that the controller includes a timing controller that controls transmission timings of ultrasonic waves so that ultrasonic waves transmitted from the plurality of ultrasonic wave transmission and reception portions are simultaneously incident to a reference plane intersecting a direction directed toward the subject.

In the application example with this configuration, the controller performs delay driving so that a timing at which an ultrasonic wave transmitted from the first ultrasonic wave transmission and reception portion passes through a reference plane intersecting (for example, orthogonal to) a direction directed toward a subject and a timing at which an ultrasonic wave transmitted from the second ultrasonic wave transmission and reception portion passes through the reference plane are the same timing. Consequently, wavefronts of ultrasonic waves transmitted from the respective ultrasonic wave transmission and reception portions can be aligned with each other, and thus it is possible to perform highly accurate ultrasonic measurement.

A control method according to an application example is a control method for the described ultrasonic unit described above, the method including performing delay driving on the plurality of ultrasonic wave transmission and reception portions so that ultrasonic waves transmitted from the plurality of ultrasonic wave transmission and reception portions are simultaneously incident to a reference plane intersecting a direction directed toward the subject.

In the application example, the controller performs delay driving so that a timing at which an ultrasonic wave transmitted from the first ultrasonic wave transmission and reception portion passes through a reference plane intersecting (for example, orthogonal to) a direction directed toward a subject and a timing at which an ultrasonic wave transmitted from the second ultrasonic wave transmission and reception portion passes through the reference plane are the same timing. Consequently, wavefronts of ultrasonic waves transmitted from the respective ultrasonic wave transmission and reception portions can be aligned with each other, and thus it is possible to perform highly accurate ultrasonic measurement.

### BRIEF DESCRIPTION OF THE DRAWINGS

The invention will be described with reference to the accompanying drawings, wherein like numbers reference like elements.

FIG. 1 is a schematic diagram illustrating a schematic configuration of an ultrasonic measurement apparatus according to a first embodiment.

FIG. 2 is a perspective view illustrating a schematic configuration of an ultrasonic probe according to the first embodiment.

FIG. 3 is a sectional view illustrating a schematic configuration when the ultrasonic probe of the first embodiment is viewed from the front.

FIG. 4 is a sectional view illustrating a schematic configuration when a main device of the first embodiment is viewed from the side.

FIG. 5 is a sectional view illustrating a schematic configuration when a first sub-device of the first embodiment is viewed from the side.

FIG. 6 is a plan view illustrating a schematic configuration of an ultrasonic substrate according to the first embodiment.

FIG. 7 is a sectional view obtained by cutting a part of the ultrasonic substrate of the first embodiment along a Y direction.

FIG. 8 is a block diagram illustrating a schematic configuration of the ultrasonic probe including a circuit substrate according to the first embodiment.

FIG. 9 is a schematic diagram schematically illustrating paths of ultrasonic waves propagating through respective sound guide portions in the first embodiment.

FIG. 10 is a diagram for explaining a delay timing during transmission of ultrasonic waves in the first embodiment.

FIG. 11 is a schematic sectional view obtained by cutting an ultrasonic probe in an XZ plane according to a third embodiment.

FIG. 12 is a schematic sectional view obtained by cutting an ultrasonic probe in an XZ plane according to a fourth embodiment.

FIG. 13 is a plan view in which an ultrasonic probe is viewed from an ultrasonic wave transmission and reception direction in Modification Example 1.

FIG. 14 is a schematic sectional view obtained by cutting an ultrasonic probe in a YZ plane according to Modification Example 1.

FIG. 15 is a schematic sectional view obtained by cutting an ultrasonic probe in a YZ plane according to Modification Example 3.

FIG. 16 is a plan view in which an ultrasonic probe is viewed from an ultrasonic wave transmission and reception direction in Modification Example 4.

### DESCRIPTION OF EXEMPLARY EMBODIMENTS

### First Embodiment

Hereinafter, a description will be made of a first embodiment of the invention.

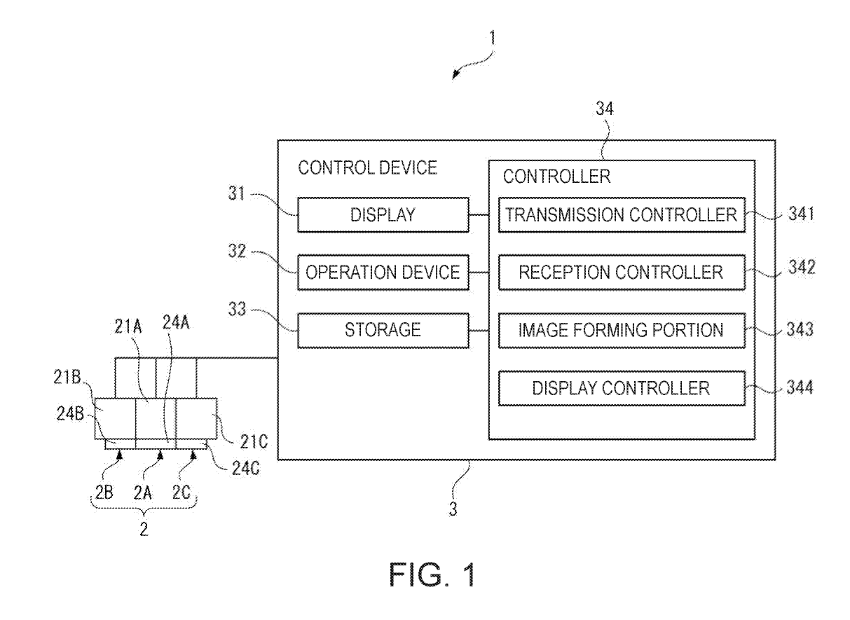

FIG. 1 is a schematic diagram illustrating a schematic configuration of an ultrasonic measurement apparatus 1 according to the present embodiment.

As illustrated in FIG. 1, the ultrasonic measurement apparatus 1 (ultrasonic apparatus) includes an ultrasonic probe 2 and a control device 3. In the ultrasonic measurement apparatus 1, the ultrasonic probe 2 is brought into contact with a subject (for example, a living body in the present embodiment), and the control device 3 controls the ultrasonic probe 2. Consequently, the ultrasonic probe 2 performs ultrasonic measurement of transmitting an ultrasonic wave to the subject, and receiving an ultrasonic wave reflected at the inside of the living body. The control device 3 receives an ultrasonic measurement result from the ultrasonic probe 2 so as to form, for example, an internal tomographic image of the subject, and displays the internal tomographic image on a display 31.

Hereinafter, each configuration of the ultrasonic measurement apparatus 1 will be described in detail.

### Configuration of Ultrasonic Probe

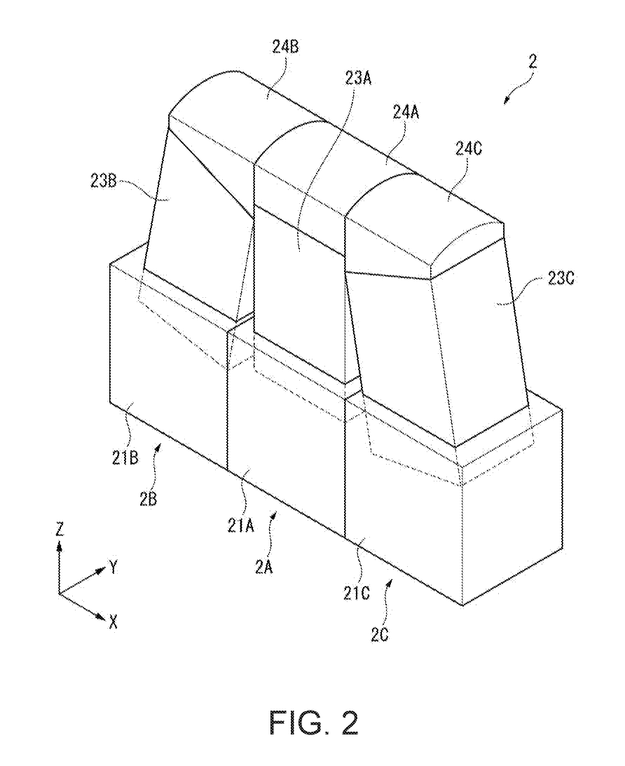

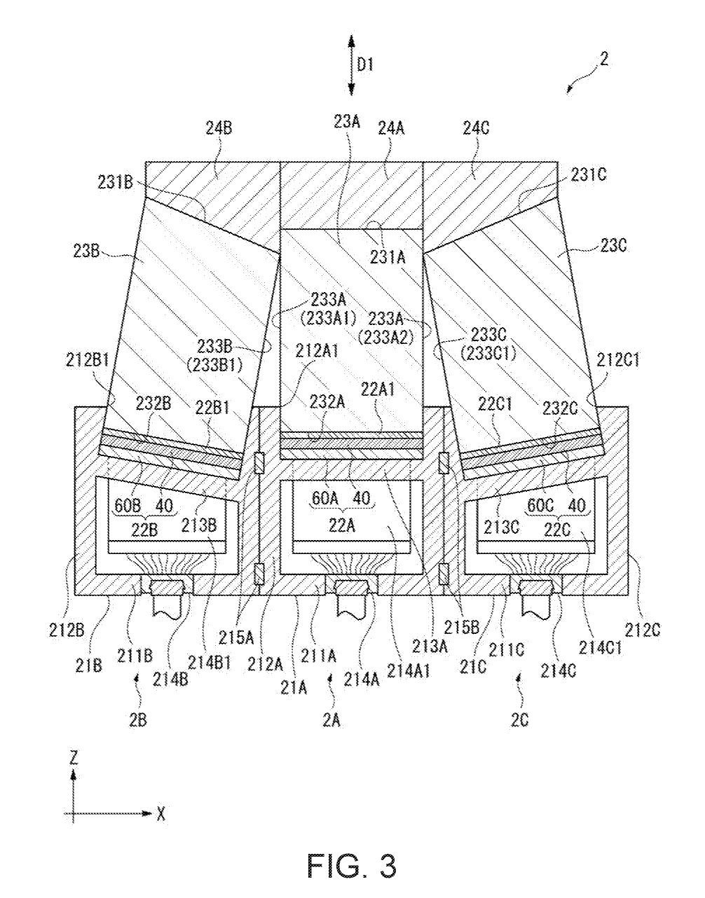

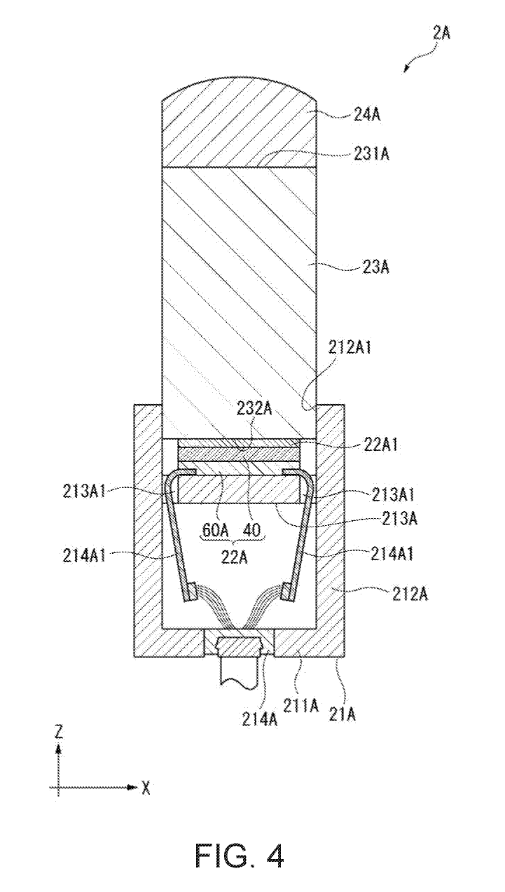

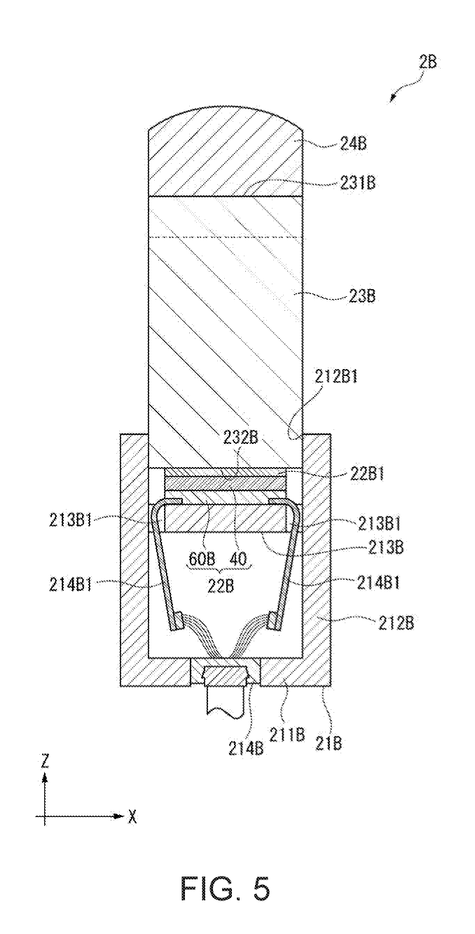

FIG. 2 is a perspective view illustrating a schematic configuration of the ultrasonic probe 2 of the present embodiment. FIG. 3 is a sectional view illustrating a schematic configuration when the ultrasonic probe 2 of the present embodiment is viewed from the front. FIG. 4 is a sectional view illustrating a schematic configuration when a main device 2A of the present embodiment is viewed from the side. FIG. 5 is a sectional view illustrating a schematic configuration when a first sub-device 2B of the present embodiment is viewed from the side.

As illustrated in FIGS. 1 to 3, the ultrasonic probe includes the main device 2A (first device), the first sub-device 2B (second device), and a second sub-device 2C (second device).

The devices 2A, 2B and 2C can separately communicate with the control device 3, and, for example, as illustrated in FIG. 1, cable wires are respectively provided in the devices 2A, 2B and 2C which are thus connected to the control device 3.

The sub-devices 2B and 2C are attachable to and detachable from the main device 2A, and can thus be attached and detached depending on a measurement location of the subject. In the present embodiment, the devices 2A, 2B and 2C are attachable and detachable along an X direction (scanning direction). In other words, the first sub-device 2B can be attached to and detached from a −X side of the main device 2A, and the second sub-device 2C can be attached to and detached from a +X side of the main device 2A.

Consequently, in a case where a wide range is a measurement range, the sub-devices 2B and 2C are attached to the main device 2A, and thus it is possible to perform ultrasonic measurement using the three devices 2A, 2B and 2C. In a case where a narrow range is a measurement range, one or both of the first sub-device 2B and the second sub-device 2C may be detached from the main device 2A and used.

As illustrated in FIGS. 3 and 4, the main device 2A is configured to include a main casing 21A, a main transmission and reception portion 22A (first ultrasonic wave transmission and reception portion), a main sound guide portion 23A (first sound guide portion), and a main acoustic lens 24A.

As illustrated in FIGS. 3 and 5, the first sub-device 2B is configured to include a first sub-casing 21B, a first sub-transmission and reception portion 22B (second ultrasonic wave transmission and reception portion), a first sub-sound guide portion 23B (second sound guide portion), and a first sub-acoustic lens 24B.

The second sub-device 2C has the substantially same configuration as that of the first sub-device 2B, and is configured to include a second sub-casing 21C, a second sub-transmission and reception portion 22C (second ultrasonic wave transmission and reception portion), a second sub-sound guide portion 23C (second sound guide portion), and a second sub-acoustic lens 24C.

An ultrasonic unit is formed of the main transmission and reception portion 22A, the main sound guide portion 23A, the first sub-transmission and reception portion 22B, the first sub-sound guide portion 23B, the second sub-transmission and reception portion 22C, and the second sub-sound guide portion 23C.

### Configuration of Casing

As illustrated in FIGS. 3 and 4, the main casing 21A includes a bottom portion 211A, a sidewall portion 212A, and a pedestal portion 213A. As illustrated in FIGS. 3 and 5, the first sub-casing 21B includes a bottom portion 211B, a sidewall portion 212B, and a pedestal portion 213B. Similarly, the second sub-casing 21C includes a bottom portion 211C, a sidewall portion 212C, and a pedestal portion 213C.

Each of the bottom portions 211A, 211B and 211C is formed, for example, in a tabular shape. A connector 214A which is connected to a cable wire via which the main device 2A is connected to the control device 3 is provided at a part of the bottom portion 211A. Similarly, a connector 214B is provided at a part of the bottom portion 211B, and a connector 214C is provided at a part of the bottom portion 211C.

The sidewall portions 212A, 212B and 212C are respectively formed to rise from outer peripheries of the bottom portions 211A, 211B and 211C, and thus form a storage space. Open windows 212A1, 212B1 and 212C1 are respectively provided on surfaces of the sidewall portions 212A, 212B and 212C opposite to the bottom portions 211A, 211B and 211C. The main sound guide portion 23A which is tightly fixed to the main transmission and reception portion 22A via an acoustic layer 22A1 is inserted into the open window 212A1 over the inside and the outside of the main casing 21A. Similarly, the first sub-sound guide portion 23B which is tightly fixed to the first sub-transmission and reception portion 22B via an acoustic layer 22B1 is inserted into the open window 212B1 over the inside and the outside of the first sub-casing 21B. The second sub-sound guide portion 23C which is tightly fixed to the second sub-transmission and reception portion 22C via an acoustic layer 22C1 is inserted into the open window 212C1 over the inside and the outside of the second sub-casing 21C.

Here, an inner peripheral surface of the sidewall portion 212A is in contact with a side surface (main side surface 233A) of the main sound guide portion 23A. Similarly, an inner peripheral surface of the sidewall portion 212B is in contact with a side surface (first sub-side surface 233B) of the first sub-sound guide portion 23B, and an inner peripheral surface of the sidewall portion 212C is in contact with a side surface (second sub-side surface 233C) of the second sub-sound guide portion 23C.

When the first sub-device 2B is attached to the main device 2A, the sidewall portion 212A and the sidewall portion 212B come into contact with each other. The sidewall portion 212A and the sidewall portion 212B are provided with fixation mechanisms 215A which attachably and detachably fix the first sub-device 2B to the main device 2A. Similarly, when the second sub-device 2C is attached to the main device 2A, the sidewall portion 212A and the sidewall portion 212C come into contact with each other. The sidewall portion 212A and the sidewall portion 212C are provided with fixation mechanisms 215B which attachably and detachably fix the second sub-device 2C to the main device 2A.

Regarding the fixation mechanisms 215A and 215B, for example, there may be a configuration in which engagement grooves are provided at one of the main casing 21A and the sub-casings 21B and 21C, and engagement claws which can be engaged with the engagement grooves are provided at the other, and fixation may be performed by using surface fasteners or the like.

Each of the pedestal portions 213A, 213B and 213C is provided at an intermediate position of the sidewall portion 212A in a height direction (a Z direction directed toward the open window 212A1 from the bottom portion 211A). The pedestal portions 213A, 213B and 213C are formed, for example, in a tabular shape. The main transmission and reception portion 22A is fixed to the pedestal portion 213A, the first sub-transmission and reception portion 22B is fixed to the pedestal portion 213B, and the second sub-transmission and reception portion 22C is fixed to the pedestal portion 213C.

Here, the pedestal portion 213A of the main casing 21A is formed in parallel to the bottom portion 211A. In contrast, the pedestal portion 213B of the first sub-casing 21B is inclined so that a normal direction thereto is tilted toward the +X side. The pedestal portion 213C of the second sub-casing 21C is inclined so that a normal direction thereto is tilted toward the −X side.

Each of the pedestal portions 213A, 213B and 213C is provided with wiring holes 213A1 and 213B1 (wiring holes of the pedestal portion 213C are not illustrated). A connection wiring 214A1 (for example, an FPC) via which the main transmission and reception portion 22A and the connector 214A are connected to each other is inserted into the wiring hole 213A1 provided in the pedestal portion 213A. A connection wiring 214B1 via which the first sub-transmission and reception portion 22B and the connector 214B are connected to each other is inserted into the wiring hole 213B1 provided in the pedestal portion 213B. A connection wiring 214C1 via which the second sub-transmission and reception portion 22C and the connector 214C are connected to each other is inserted into the wiring hole provided in the pedestal portion 213C.

### Configuration of Ultrasonic Wave Transmission and Reception Portion

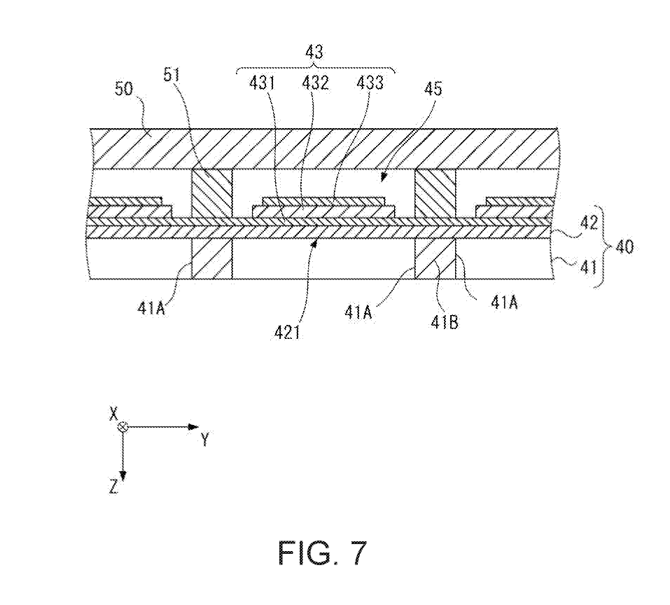

The main transmission and reception portion 22A, the first sub-transmission and reception portion 22B, and the second sub-transmission and reception portion 22C are members (ultrasonic wave transmission and reception portion) transmitting and receiving ultrasonic waves. The main transmission and reception portion 22A, the first sub-transmission and reception portion 22B, and the second sub-transmission and reception portion 22C have the same ultrasonic wave transmission and reception structure, and, for example, each thereof is configured to include an ultrasonic substrate 40, a sealing plate 50 (refer to FIG. 7), and a circuit substrate. In the present embodiment, as illustrated in FIG. 3, the main transmission and reception portion 22A, the first sub-transmission and reception portion 22B, and the second sub-transmission and reception portion 22C are disposed at positions separated from each other by a predetermined distance in the X direction.

Hereinafter, a description will be made of the ultrasonic substrate 40, the sealing plate 50, and the circuit substrate forming the main transmission and reception portion 22A, the sub-transmission and reception portions 22B and 22C.

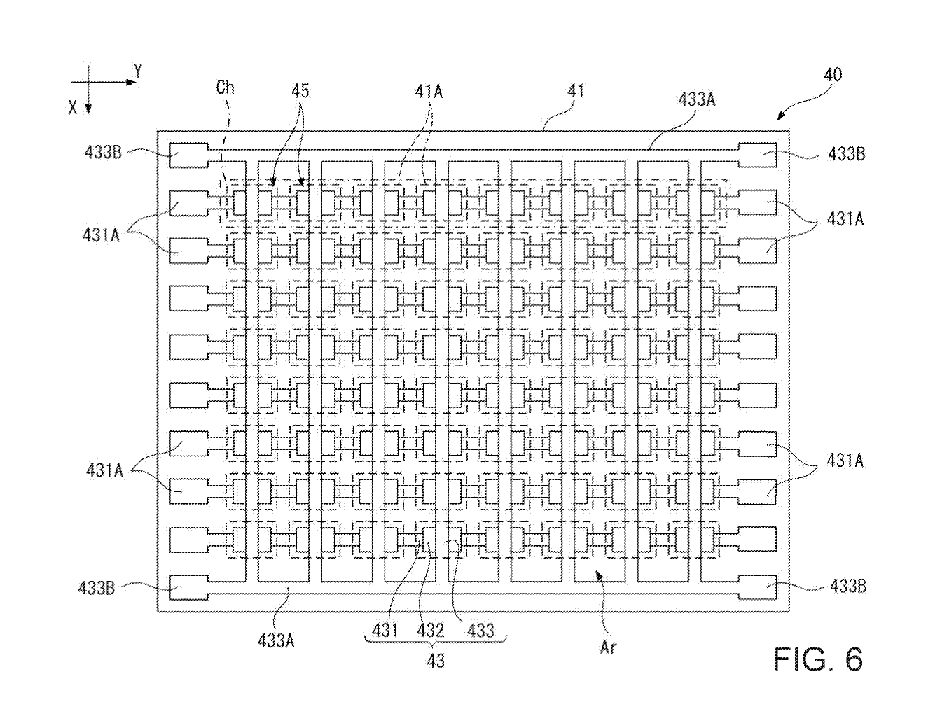

FIG. 6 is a plan view illustrating a schematic configuration of the ultrasonic substrate 40 of the present embodiment.

As illustrated in FIG. 6, a plurality of ultrasonic transducers 45 (ultrasonic elements) are disposed in a two-dimensional array form along the X direction (scanning direction) and the Y direction (slice direction) which intersect each other (are orthogonal to each other as an example in the present embodiment) on the ultrasonic substrate 40. Here, a transmission and reception train Ch (element group) of one channel (CH) is formed by a plurality of ultrasonic transducers 45 disposed in the Y direction. The transmission and reception train Ch of one CH is disposed to be arranged in a plurality along the Y direction, and thus the ultrasonic substrate 40 having a one-dimensional array structure is formed. Here, a region in which the ultrasonic transducers 45 are disposed is referred to as an array region Ar.

In FIG. 6, for convenience of description, the number of disposed ultrasonic transducers 45 is reduced, but, actually, a larger number of ultrasonic transducers 45 are disposed.

FIG. 7 is a sectional view obtained by cutting the ultrasonic substrate 40 along the Y direction.

As illustrated in FIG. 7, the ultrasonic substrate 40 is configured to include an element base plate 41, a support film 42 provided on the element base plate 41, and a piezoelectric element 43 provided on the support film 42.

The element base plate 41 is formed of, for example, a semiconductor base plate such as Si. The element base plate 41 is provided with an opening portion 41A corresponding to each ultrasonic transducer 45. In the present embodiment, each opening portion 41A is a through-hole which penetrates through the element base plate 41 in a base plate thickness direction, and the support film 42 is provided at one end side (sealing plate 50 side) of the through-hole.

An acoustic layer fills a side of the opening portion 41A on which the support film 42 is not provided. The acoustic layer corresponds to the acoustic layer 22A1 illustrated in FIGS. 3 and 4 in the main transmission and reception portion 22A, corresponds to the acoustic layer 22B1 as illustrated in FIGS. 3 and 5 in the first sub-transmission and reception portion 22B, and corresponds to the acoustic layer 22C1 illustrated in FIG. 3 in the second sub-transmission and reception portion 22C.

The support film 42 is formed of, for example, a laminate of SiO₂ and ZrO₂, and is provided to cover the entire element base plate 41 on the sealing plate 50 side. In other words, the support film 42 is supported by a partition wall 41B forming the opening portion 41A, and closes the opening portion 41A on the sealing plate 50 side. A thickness dimension of the support film 42 is sufficiently smaller than a thickness dimension of the element base plate 41.

The piezoelectric element 43 is provided on the support film 42 closing each opening portion 41A. The piezoelectric element 43 is formed of a laminate obtained by laminating a lower electrode 431, a piezoelectric film 432, and an upper electrode 433 from the support film 42 side.

Here, a portion of the support film 42 closing the opening portion 41A forms a vibration portion 421, and a single ultrasonic transducer 45 is formed of the vibration portion 421 and the piezoelectric element 43.

In the ultrasonic transducer 45, a square wave voltage (drive signal) having a predetermined frequency is applied between the lower electrode 431 and the upper electrode 433, and thus the piezoelectric film 432 is bent so that the vibration portion 421 vibrates to send an ultrasonic wave. If the vibration portion 421 vibrates due to an ultrasonic wave reflected from a living body, a potential difference is generated between the upper and lower parts of the piezoelectric film 432. Consequently, a potential difference generated between the lower electrode 431 and the upper electrode 433 is detected, and thus the received ultrasonic wave can be detected.

In the present embodiment, as illustrated in FIG. 6, the lower electrode 431 is formed linearly along the Y direction, and connects a plurality of ultrasonic transducers 45 forming the transmission and reception train Ch of one CH to each other. Drive terminals 431A are provided at both ends of the lower electrode 431. Each of the drive terminals 431A is electrically connected to the circuit substrate.

The upper electrode 433 is formed linearly along the X direction, and connects the ultrasonic transducers 45 arranged in the X direction to each other. Ends of the upper electrode 433 on the ±X sides are connected to common electrode lines 433A. The common electrode line 433A connects the upper electrodes 433 disposed in a plurality along the Y direction to each other, and a common terminal 433B electrically connected to the circuit substrate is provided at an end thereof.

The sealing plate 50 is formed so that a planar shape viewed from a thickness direction is the same as, for example, a shape of the ultrasonic substrate 40, and is fixed to the support film 42 side of the ultrasonic substrate 40 via, for example, a fixation member 51 such as a resin so as to reinforce the ultrasonic substrate 40.

The sealing plate 50 is provided with opening portions (not illustrated) at positions facing the drive terminal 431A and the common terminal 433B of the element base plate 41, and the drive terminal 431A and the common terminal 433B are electrically connected to the circuit substrate via the opening portions by using, for example, an FPC. Through-electrodes which penetrate through the sealing plate 50 in the thickness direction may be provided, and the drive terminal 431A and the common terminal 433B may be electrically connected to the circuit substrate via the through-electrodes.

The circuit substrate is connected to the drive terminal 431A or the common terminal 433B. The circuit substrate is connected to the control device 3, and controls driving of the ultrasonic substrate 40 on the basis of a command signal for an instruction for ultrasonic wave transmission and reception processes from the control device 3.

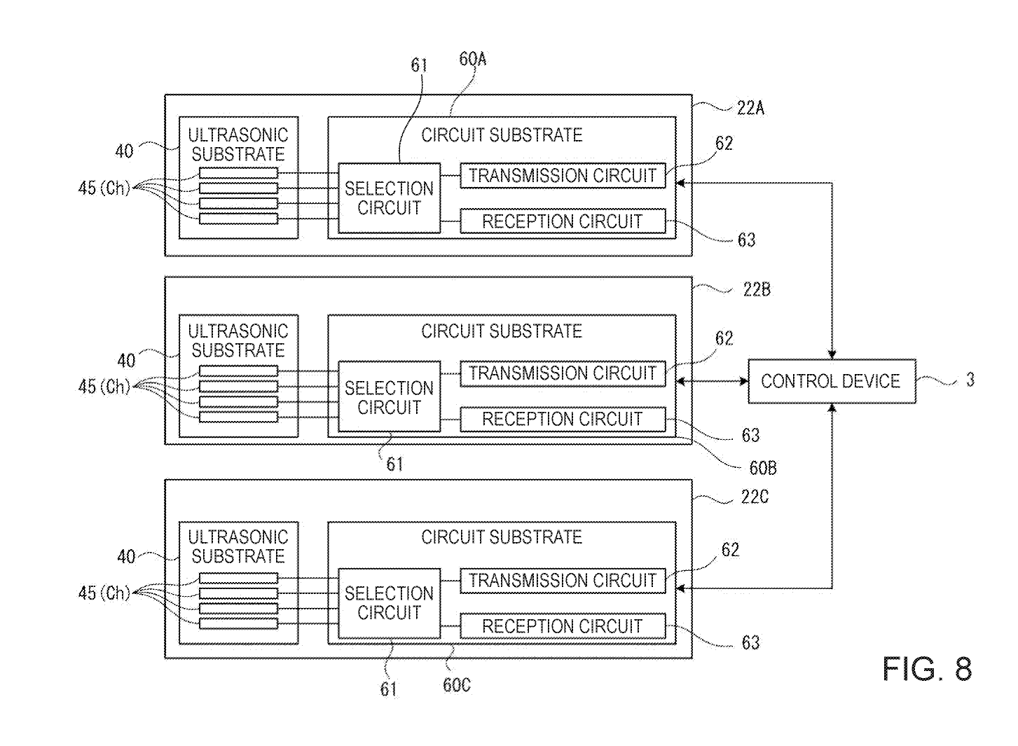

FIG. 8 is a block diagram illustrating a schematic configuration of the ultrasonic probe 2 including the circuit substrate of the present embodiment.

In the present embodiment, as illustrated in FIG. 8, there are provided a main circuit substrate 60A for controlling the ultrasonic substrate 40 of the main transmission and reception portion 22A, a first sub-circuit substrate 60B for controlling the ultrasonic substrate 40 of the first sub-transmission and reception portion 22B, and a second sub-circuit substrate 60C for controlling the ultrasonic substrate 40 of the second sub-transmission and reception portion 22C.

Each of the circuit substrates 60A, 60B and 60C includes, as illustrated in FIG. 8, for example, a selection circuit 61, a transmission circuit 62, and a reception circuit 63 as various circuits driving the ultrasonic transducer 45. Each of the circuit substrates 60A, 60B and 60C may include a ground circuit or the like which maintains the common terminal 433B at a predetermined common potential.

The selection circuit 61 switches between transmission connection for connection between the ultrasonic transducers 45 (transmission and reception train Ch) and the transmission circuit 62, and reception connection for connection between the ultrasonic transducers 45 (transmission and reception train Ch) and the reception circuit 63, under the control of the control device 3.

The transmission circuit 62 outputs a transmission signal for transmitting an ultrasonic wave, to each of the ultrasonic transducers 45 (each transmission and reception train Ch) via the selection circuit 61 when switching to the transmission connection occurs under the control of the control device 3.

The reception circuit 63 outputs a received signal which is input from the ultrasonic transducers 45 (transmission and reception train Ch) via the selection circuit 61 to the control device 3 when switching to the reception connection occurs under the control of the control device 3. The reception circuit 63 is configured to include, for example, a low noise amplification circuit, a voltage controlled attenuator, a programmable gain amplifier, a low-pass filter, and an A/D converter, and performs signal processes such as conversion of the received signal to a digital signal, removal of a noise component, and amplification to a desired signal level, and the received signal having undergone the processes to the control device 3.

### Configuration of Sound Guide Portion

Returning to FIGS. 2 to 5, the main sound guide portion 23A is provided on the main transmission and reception portion 22A via the acoustic layer 22A1. The first sub-sound guide portion 23B is provided on the first sub-transmission and reception portion 22B via the acoustic layer 22B1. The second sub-sound guide portion 23C is provided on the second sub-transmission and reception portion 22C via the acoustic layer 22C1. The sound guide portions 23A, 23B and 23C are made of, for example, silicone, and thus have the substantially same acoustic impedance as that of a living body. An ultrasonic wave transmitted from each of the ultrasonic wave transmission and reception portions 22A, 22B and 22C can be uniformly transmitted in a predetermined first ultrasonic wave transmission and reception direction by the main sound guide portion 23A, the first sub-sound guide portion 23B, and the second sub-sound guide portion 23C, and thus an ultrasonic wave which is incident from the ultrasonic wave transmission and reception direction can be guided to each of the ultrasonic wave transmission and reception portions 22A, 22B and 22C so as to be received.

The main sound guide portion 23A is formed in a substantially rectangular parallelepiped shape having a distal end surface (main distal end surface 231A), a basal end surface (main basal end surface 232A), and a side surface (main side surface 233A).

The main distal end surface 231A is a surface on which the main acoustic lens 24A is provided, and is a planar surface which is parallel to the pedestal portion 213A of the main casing 21A.

The main basal end surface 232A is a surface (coupling surface) which is connected to the main transmission and reception portion 22A via the acoustic layer 22A1 on an opposite side to the main distal end surface 231A, and is a planar surface which is parallel to the pedestal portion 213A of the main casing 21A.

The main side surface 233A is a side surface via which the main distal end surface 231A is connected to the main basal end surface 232A, and is a planar surface which is orthogonal to the main distal end surface 231A and the main basal end surface 232A. Here, a surface on the first sub-device 2B side (−X side) will be referred to as a first main side surface 233A1, and a surface on the second sub-device 2C side (+X side) will be referred to as a second main side surface 233A2.

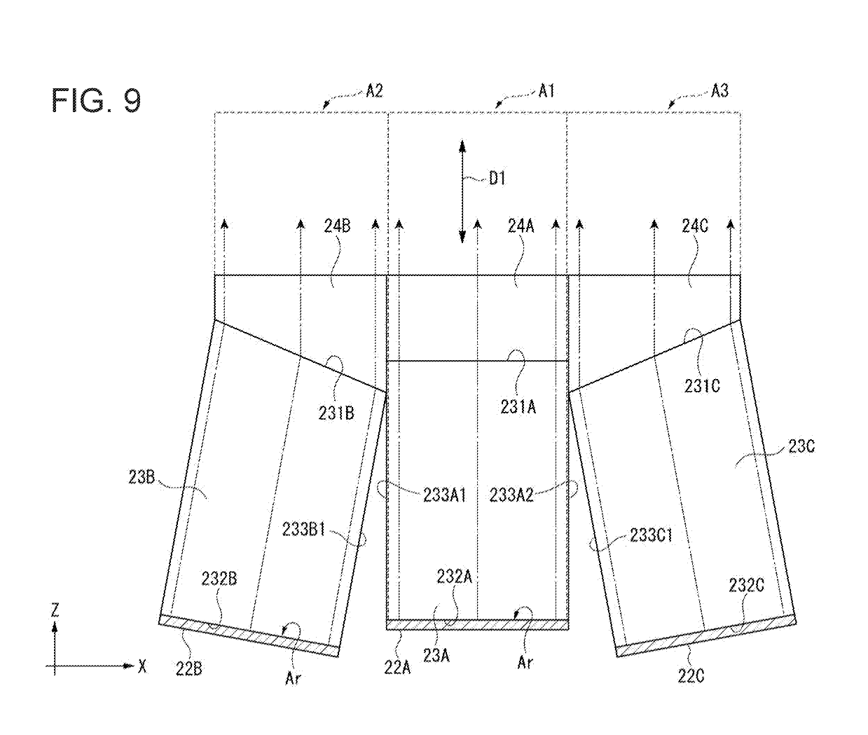

FIG. 9 is a schematic diagram schematically illustrating paths of ultrasonic waves propagating through the respective sound guide portions (the main sound guide portion 23A, the first sub-sound guide portion 23B, and the second sub-sound guide portion 23C).

The main basal end surface 232A of the main sound guide portion 23A is preferably formed in the same shape and with the same size as those of the array region Ar (a region in which the ultrasonic transducers 45 are disposed in an array form) in the main transmission and reception portion 22A. The main distal end surface 231A is also preferably formed in the same shape and with the same size as those of the array region Ar. The main distal end surface 231A and the main basal end surface 232A overlap and match each other in a projection view of the main sound guide portion 23A from a normal direction to the main distal end surface 231A and the main basal end surface 232A.

In the main sound guide portion 23A with the above-described configuration, an ultrasonic wave transmitted from the array region Ar of the main transmission and reception portion 22A propagates toward the main distal end surface 231A from the main basal end surface 232A, and is sent in a normal direction (ultrasonic wave transmission and reception direction D1; a direction toward a living body corresponding to a subject) to the main distal end surface 231A.

The first sub-sound guide portion 23B is configured to include a distal end surface (first sub-distal end surface 231B), a basal end surface (first sub-basal end surface 232B), and a side surface (first sub-side surface 233B).

The first sub-distal end surface 231B is a surface on which the first sub-acoustic lens 24B is provided. The first sub-basal end surface 232B is a surface (coupling surface) which is connected to the first sub-transmission and reception portion 22B via the acoustic layer 22B1 on an opposite side to the first sub-distal end surface 231B, and is inclined with respect to the main basal end surface 232A (two surfaces are different planar surfaces).

Here, the first sub-basal end surface 232B is a planar surface which is parallel to the pedestal portion 213B of the first sub-casing 21B. On the other hand, the first sub-distal end surface 231B is a planar surface which is inclined with respect to the first sub-basal end surface 232B. Specifically, the first sub-distal end surface 231B is inclined so that a distance between the first sub-distal end surface 231B and the first sub-basal end surface 232B is reduced toward the +X side. As illustrated in FIG. 9, an inclined angle of the first sub-distal end surface 231B is an angle at which an ultrasonic wave is emitted in the ultrasonic wave transmission and reception direction D1 when the ultrasonic wave sent from the first sub-transmission and reception portion 22B passes through the first sub-distal end surface 231B. Such an inclined angle is set as appropriate according to a difference or the like between sonic speeds in the first sub-sound guide portion 23B and the first sub-acoustic lens 24B.

The first sub-basal end surface 232B is preferably formed in the same shape and with the same size as those of the array region Ar of the first sub-transmission and reception portion 22B. The first sub-distal end surface 231B preferably has a shape and a size so as to match the first sub-basal end surface 232B in a projection view of the first sub-sound guide portion 23B from a normal direction to the first sub-basal end surface 232B.

An end of the first sub-distal end surface 231B on the +X side is in contact with the first main side surface 233A1 of the main sound guide portion 23A. Therefore, there is a configuration in which an ultrasonic wave emission surface (main distal end surface 231A) of an ultrasonic wave sent by the main transmission and reception portion 22A is adjacent to an ultrasonic wave emission surface (first sub-distal end surface 231B) of an ultrasonic wave sent by the first sub-transmission and reception portion 22B when viewed from the ultrasonic wave transmission and reception direction D1.

In other words, in the present embodiment, a distance between the main distal end surface 231A via which an ultrasonic wave from the main transmission and reception portion 22A is input and output and the first sub-distal end surface 231B via which an ultrasonic wave from the first sub-transmission and reception portion 22B is input and output is shorter than a distance between the main transmission and reception portion 22A and the first sub-transmission and reception portion 22B. In this case, as illustrated in FIG. 9, a transmission and reception range A1 (a range in which an ultrasonic wave can be transmitted and received) in which an ultrasonic wave from the main transmission and reception portion 22A is sent is close to a transmission and reception range A2 (a range in which an ultrasonic wave can be transmitted and received) in which an ultrasonic wave from the first sub-transmission and reception portion 22B is sent, and thus the ultrasonic wave can be uniformly sent (a gap between the transmission and reception range A1 and the transmission and reception range A2 is reduced).

The first sub-side surface 233B is a side surface via which the first sub-distal end surface 231B is connected to the first sub-basal end surface 232B. Here, a surface on the main sound guide portion 23A side (+X side) will be referred to as a first sub-facing side surface 233B1.

In the present embodiment, as described above, a +X side end edge of the first sub-distal end surface 231B is in contact with the first main side surface 233A1 of the main sound guide portion 23A. The first sub-side surface 233B is a surface which is perpendicular to the first sub-basal end surface 232B. Therefore, the first sub-facing side surface 233B1 is inclined with respect to the first main side surface 233A1, and thus forms an air layer (intermediate portion) with the first main side surface 233A1. In other words, a distance between the main sound guide portion 23A and the first sub-sound guide portion 23B is gradually reduced toward a subject (living body). Therefore, the transmittance of an ultrasonic wave of the distal end surface (the main distal end surface 231A and the first sub-distal end surface 231B) side is higher than the transmittance of an ultrasonic wave of the basal end surface (the main basal end surface 232A and the first sub-basal end surface 232B) side in the surface along the ultrasonic wave transmission and reception direction D1 between the main transmission and reception portion 22A and the first sub-transmission and reception portion 22B.

This configuration prevents a defect that an ultrasonic wave sent from the main transmission and reception portion 22A is incident to the first sub-sound guide portion 23B side from the main basal end surface 232A side of the main sound guide portion 23A so as to cause crosstalk. This configuration prevents a defect that an ultrasonic wave sent from the first sub-transmission and reception portion 22B is incident to the main sound guide portion 23A side from the first sub-basal end surface 232B side of the first sub-sound guide portion 23B so as to cause crosstalk.

A main component of an ultrasonic wave which is incident from the ultrasonic wave transmission and reception direction D1 is refracted toward the main transmission and reception portion 22A or the first sub-transmission and reception portion 22B by the main distal end surface 231A or the first sub-distal end surface 231B. However, some components of the incident ultrasonic wave advance toward between the main transmission and reception portion 22A and the first sub-transmission and reception portion 22B. Also in this case, in the present embodiment, as described above, the air layer is interposed between the first main side surface 233A1 and the first sub-facing side surface 233B1, and thus a direction of the incident ultrasonic wave can be corrected to the main transmission and reception portion 22A side or the first sub-transmission and reception portion 22B side. Consequently, reception sensitivity in ultrasonic wave reception is made favorable.

The second sub-sound guide portion 23C is configured to include a second sub-distal end surface 231C, a second sub-basal end surface 232C, and a second sub-side surface 233C (a surface facing the second main side surface 233A2 will be referred to as a second sub-facing side surface 233C1). In the second sub-sound guide portion 23C, an inclined direction of the second sub-distal end surface 231C is reverse to that of the first sub-distal end surface 231B. In other words, the second sub-distal end surface 231C is inclined so that a distance between the second sub-distal end surface 231C and the second sub-basal end surface 232C is reduced toward the −X side.

An end of the second sub-distal end surface 231C on the −X side is in contact with the second main side surface 233A2 of the main sound guide portion 23A, and the second sub-facing side surface 233C1 is inclined with respect to the second main side surface 233A2.

Other configurations are the same as those of the first sub-sound guide portion 23B, and thus description thereof will be omitted here.

As illustrated in FIG. 9, by using the second sub-sound guide portion 23C, the transmission and reception range A1 (a range in which an ultrasonic wave can be transmitted and received) in which an ultrasonic wave from the main transmission and reception portion 22A is sent is close to a transmission and reception range A3 (a range in which an ultrasonic wave can be transmitted and received) in which an ultrasonic wave from the second sub-transmission and reception portion 22C is sent, and thus the ultrasonic wave can be uniformly sent (a gap between the transmission and reception range A1 and the transmission and reception range A3 is reduced).

### Configuration of Acoustic Lens

The main acoustic lens 24A is in close contact with and fixed to the main distal end surface 231A of the main sound guide portion 23A. The first sub-acoustic lens 24B is in close contact with and fixed to the first sub-distal end surface 231B of the first sub-sound guide portion 23B. The second sub-acoustic lens 24C is in close contact with and fixed to the second sub-distal end surface 231C of the second sub-sound guide portion 23C.

Each of the acoustic lenses 24A, 24B and 24C has a substantially cylindrical shape with the X direction as an axis. In other words, when cut in a YZ plane, surface shapes of the acoustic lenses 24A, 24B and 24C on opposite sides to the sound guide portions 23A, 23B and 23C are circular arc shapes.

In the X direction, the side surface of the main acoustic lens 24A on the first sub-acoustic lens 24B side is in close contact with the side surface of the first sub-acoustic lens 24B on the main acoustic lens 24A side. Similarly, the side surface of the main acoustic lens 24A on the second sub-acoustic lens 24C side is in close contact with the side surface of the second sub-acoustic lens 24C on the main acoustic lens 24A side.

Here, each of the acoustic lenses 24A, 24B and 24C has an acoustic impedance close to that of a living body which is a subject, and has an acoustic impedance lower than that of each of the sound guide portions 23A, 23B and 23C. Consequently, transmission directions of ultrasonic waves can be aligned with the ultrasonic wave transmission and reception direction D1 when an ultrasonic wave propagates toward the first sub-acoustic lens 24B from the first sub-distal end surface 231B of the first sub-sound guide portion 23B and when an ultrasonic wave propagates toward the second sub-acoustic lens 24C from the second sub-distal end surface 231C.

### Configuration of Control Device

As illustrated in FIG. 1, the control device 3 is configured to include, for example, the display 31, an operation device 32, a storage 33, and a controller 34. As the control device 3, for example, a terminal device such as a tablet terminal, a smart phone, or a personal computer may be used, and a dedicated terminal device for operating the ultrasonic probe 2 may be used.

The display 31 is formed of, for example, a liquid crystal display, and displays an image.

The operation device 32 is a user interface (UI) for a user operating the ultrasonic measurement apparatus 1, and may be formed of, for example, a touch panel provided on the display 31, an operation button, a keyboard, or a mouse.

The storage 33 stores various programs and various pieces of data for controlling the ultrasonic measurement apparatus 1.

The controller 34 is formed of, for example, a calculation circuit such as a central processing unit (CPU), and a storage circuit such as a memory. The controller 34 reads the various programs stored in the storage 33 and executes the programs, so as to function as a transmission controller 341, a reception controller 342, an image forming portion 343, and a display controller 344, and controls the ultrasonic probe 2.

The transmission controller 341 outputs a command signal for switching to transmission connection and a command signal for outputting a drive signal to the transmission circuit 62, to the selection circuit 61 of each of the circuit substrates 60A, 60B and 60C.

The reception controller 342 outputs a command signal for switching to reception connection and a command signal for causing the reception circuit 63 to perform a reception process on a received signal, to the selection circuit 61 of each of the circuit substrates 60A, 60B and 60C.

The image forming portion 343 processes a received signal which is received from the reception circuit 63, so as to generate an internal tomographic image of a living body.

The display controller 344 controls the display 31 to display the generated internal tomographic image.

### Control Method for Ultrasonic Unit

In the present embodiment, if an input signal for performing ultrasonic measurement using the ultrasonic probe 2 is input to the control device 3, the transmission controller 341 of the control device 3 outputs a command signal for outputting a drive signal at a timing corresponding to a position of the ultrasonic wave transmission and reception portion, to the respective ultrasonic transducers 45 (transmission and reception train Ch). In other words, the transmission controller 341 forms a timing controller.

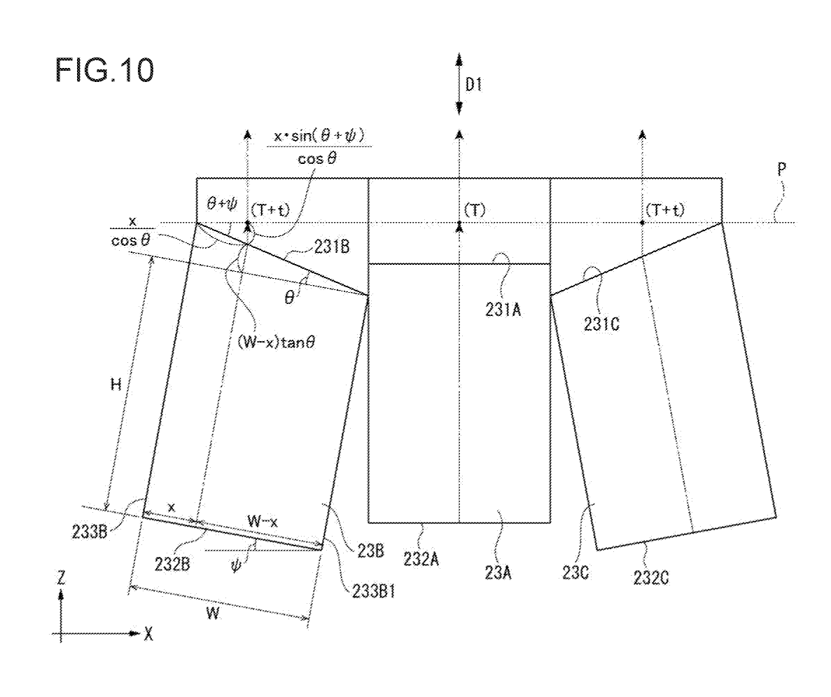

FIG. 10 is a diagram for explaining a delay timing during transmission of an ultrasonic wave.

In a case where a plane passing through tip parts of the first sub-sound guide portion 23B and the second sub-sound guide portion 23C on the ultrasonic wave transmission and reception direction D1 side is set as a reference plane P (refer to FIG. 10; the reference plane P which is orthogonal to the ultrasonic wave transmission and reception direction D1), the transmission controller 341 delays drive timings (ultrasonic wave transmission timings) of the respective ultrasonic transducers 45 (transmission and reception train Ch) so that ultrasonic waves output from the respective ultrasonic transducers 45 of the main transmission and reception portion 22A, the first sub-transmission and reception portion 22B, and the second sub-transmission and reception portion 22C simultaneously pass through the reference plane P.

Here, an inclined angle of the first sub-transmission and reception portion 22B with respect to the main transmission and reception portion 22A is indicated by ψ, an inclined angle of the first sub-distal end surface 231B with respect to the first sub-basal end surface 232B is indicated by θ, a width dimension of the array region Ar along the X direction (scanning direction) is indicated by W, and a dimension from the first sub-distal end surface 231B to the first sub-basal end surface 232B in the first sub-facing side surface 233B1 is indicated by H. The time required for an ultrasonic wave output from each ultrasonic transducer 45 of the main transmission and reception portion 22A to reach the reference plane P is indicated by T.

In the first sub-transmission and reception portion 22B, an ultrasonic wave sent from the ultrasonic transducer 45 located at a distance x from a −X side end of the array region Ar is incident to a position of the distance x from the first sub-side surface 233B on an opposite side to the first sub-facing side surface 233B1 in the first sub-basal end surface 232B. The ultrasonic wave which is incident to the first sub-basal end surface 232B advances at a sonic speed c1 corresponding to a material of the first sub-sound guide portion 23B over a distance of H+(W−x)tan θ from the first sub-distal end surface 231B to emission. An ultrasonic wave emitted from the first sub-distal end surface 231B advances at a sonic speed c2 corresponding to a material of the first sub-acoustic lens 24B over a distance of (x·sin (θ+ψ))/cos θ to the reference plane P.

Therefore, the transmission controller 341 delays a drive timing by a delay time t in the following Equation (1) from a drive time of each ultrasonic transducer 45 (transmission and reception train Ch) of the main transmission and reception portion 22A, and drives the ultrasonic transducers 45 (transmission and reception train Ch) located at the distance x from the −X side end of the array region Ar of the first sub-transmission and reception portion 22B. In a case where a value of t is a positive value, the ultrasonic transducer 45 is driven at a timing which is later than, by the time t, a drive timing of each ultrasonic transducer 45 of the main transmission and reception portion 22A. In a case where a value of t is a negative value, the ultrasonic transducer 45 is driven at a timing which is earlier than, by the time t, a drive timing of each ultrasonic transducer 45 of the main transmission and reception portion 22A.

$$t = T - ( \frac{H + ( W - x ) tan θ}{c 1} + \frac{x \cdot sin ( θ + ϕ )}{c 2 \cdot cos θ} ) \qquad ( 1 )$$

The above-described delay time is a delay time regarding the first sub-transmission and reception portion 22B, but is also the same for the second sub-transmission and reception portion 22C. In the second sub-transmission and reception portion 22C, the ultrasonic transducer 45 at the distance x from the +X side end of the array region Ar is driven at the delay time t based on the above Equation (1), and thus timings at which ultrasonic waves reach the reference plane P can be aligned with each other.

Regarding received ultrasonic waves which are received by the respective ultrasonic transducers 45 (transmission and reception train Ch), acoustic distances in the first sub-sound guide portion 23B and the second sub-sound guide portion 23C are different from each other. Therefore, the reception controller 342 delays a reception timing of a received signal by taking into consideration the delay time t shown in Equation (1). For example, in a case where a drive timing is delayed by the time t in ultrasonic wave transmission, a timing which is delayed by the time t from the timing at which a received signal is output is set as a reception timing. In a case where a drive timing is advanced by the time t in ultrasonic wave transmission, a timing which is advanced by the time t from the timing at which a received signal is output is set as a reception timing.

### Advantageous Effects of First Embodiment

In the present embodiment, the ultrasonic probe 2 includes the main sound guide portion 23A through which ultrasonic waves transmitted and received in the main transmission and reception portion 22A pass; the first sub-sound guide portion 23B through which ultrasonic waves transmitted and received in the first sub-transmission and reception portion 22B pass; and the second sub-sound guide portion 23C through which ultrasonic waves transmitted and received in the second sub-transmission and reception portion 22C pass.

A distance between the main sound guide portion 23A and the first sub-sound guide portion 23B is gradually reduced toward a subject, and a distance between the main sound guide portion 23A and the second sub-sound guide portion 23C is also reduced toward the subject. In other words, a distance between the main distal end surface 231A and the first sub-distal end surface 231B is shorter than a distance between the main transmission and reception portion 22A and the first sub-transmission and reception portion 22B, and a distance between the main distal end surface 231A and the second sub-distal end surface 231C is shorter than a distance between the main transmission and reception portion 22A and the second sub-transmission and reception portion 22C.

Thus, even in a case where the main transmission and reception portion 22A is separated from the first sub-transmission and reception portion 22B (second sub-transmission and reception portion 22C), the transmission and reception range A1 in which ultrasonic measurement can be performed by the main transmission and reception portion 22A is close to the transmission and reception range A2 in which ultrasonic measurement can be performed by the first sub-transmission and reception portion 22B (the transmission and reception range A3 in which ultrasonic measurement can be performed by the second sub-transmission and reception portion 22C). Therefore, even in a case where there is a region in which transmission and reception of ultrasonic waves cannot be performed between the main transmission and reception portion 22A and the first sub-transmission and reception portion 22B or between the main transmission and reception portion 22A and the second sub-transmission and reception portion 22C, it is possible to uniformly transmit and receive ultrasonic waves in the ultrasonic probe 2. Consequently, when an internal tomographic image is formed by the image forming portion 343, it is possible to prevent a defect such as the presence of a region in which ultrasonic measurement is not performed at a part of an internal tomographic image, and thus to form an accurate internal tomographic image.

In a case where the main transmission and reception portion 22A, the first sub-transmission and reception portion 22B, and the second sub-transmission and reception portion 22C are provided on a single substrate, as a range in which ultrasonic measurement is performed is increased, a substrate size is required to be also increased, and thus apparatus cost increases. In contrast, in the present embodiment, the ultrasonic substrate 40 having the main transmission and reception portion 22A and the ultrasonic substrate 40 having the first sub-transmission and reception portion 22B can be formed separately from each other, and thus apparatus cost can be reduced.

In the present embodiment, the main distal end surface 231A and the first sub-distal end surface 231B (second sub-distal end surface 231C) are adjacent to each other when viewed from the ultrasonic wave transmission and reception direction D1. Thus, the transmission and reception range A1 in which ultrasonic measurement can be performed by the main transmission and reception portion 22A is adjacent to the transmission and reception range A2 in which ultrasonic measurement can be performed by the first sub-transmission and reception portion 22B (the transmission and reception range A3 in which ultrasonic measurement can be performed by the second sub-transmission and reception portion 22C). In other words, a measurement impossible region in which transmission and reception of ultrasonic waves are not performed is not present between the transmission and reception range A1 and the transmission and reception range A2 (transmission and reception range A3). Consequently, it is possible to perform ultrasonic measurement with higher accuracy.

In the present embodiment, there is a gap between the main sound guide portion 23A and the first sub-sound guide portion 23B (second sub-sound guide portion 23C), and an air layer (intermediate portion) is formed therein.

Air has an acoustic impedance which is considerably lower than an acoustic impedance of the main sound guide portion 23A, the first sub-sound guide portion 23B, and the second sub-sound guide portion 23C, and thus an ultrasonic wave is reflected at an interface between the sound guide portions 23A, 23B and 23C and air. Therefore, it is possible to prevent propagation of an ultrasonic wave from the main sound guide portion 23A to the first sub-sound guide portion 23B (second sub-sound guide portion 23C) or from the first sub-sound guide portion 23B (second sub-sound guide portion 23C) to the main sound guide portion 23A, and thus to minimize crosstalk.

The first sub-facing side surface 233B1 or the second sub-facing side surface 233C1 is inclined with respect to the main side surface 233A which is parallel to the ultrasonic wave transmission and reception direction D1.

When an ultrasonic wave is incident to the first sub-sound guide portion 23B or the second sub-sound guide portion 23C from the ultrasonic wave transmission and reception direction D1, the ultrasonic wave is refracted at an angle corresponding to an inclined angle of the first sub-distal end surface 231B or the second sub-distal end surface 231C, and is directed toward the first sub-transmission and reception portion 22B or the second sub-transmission and reception portion 22C. However, some ultrasonic wave components advance straight along the ultrasonic wave transmission and reception direction D1 without being refracted at the first sub-distal end surface 231B or the second sub-distal end surface 231C. In contrast, in the present embodiment, even in a case where such some ultrasonic wave components are present, some ultrasonic wave components can be reflected in an inclined direction of the first sub-facing side surface 233B1 or the second sub-facing side surface 233C1. Consequently, it is possible to increase the sound pressure of an ultrasonic wave received at the first sub-transmission and reception portion 22B or the second sub-transmission and reception portion 22C.

In the present embodiment, the sub-devices 2B and 2C are attachable to and detachable from the main device 2A. Thus, in a case where ultrasonic measurement is desired to be performed on a region in a narrow range such as a gap between ribs, the sub-devices 2B and 2C can be detached, and ultrasonic measurement can be performed by using only the main device 2A. In a case where ultrasonic measurement is performed on a region in a wide range such as the abdomen, the sub-devices 2B and 2C can be attached to the main device 2A, and ultrasonic measurement using the three devices can be performed.

In the present embodiment, the ultrasonic substrate 40 on which the main transmission and reception portion 22A is provided and the ultrasonic substrates 40 on which the sub-transmission and reception portions 22B and 22C are provided are disposed on different planes.

Also in this case, in the present embodiment, the ultrasonic wave transmission and reception ranges A1 to A3 for the respective ultrasonic wave transmission and reception portions can be made close to each other. A plane on which the ultrasonic substrate 40 is disposed may be set as appropriate according to an arrangement relationship among other members of the devices 2A, 2B and 2C. In other words, it is possible to improve a degree of freedom of design of each of the devices 2A, 2B and 2C compared with the respective ultrasonic substrates 40 are disposed on the same plane.

In the present embodiment, the control device 3 includes the controller 34 controlling the ultrasonic probe 2, and thus can form an internal tomographic image of a living body and display the internal tomographic image on the display 31 on the basis of an ultrasonic measurement result in the ultrasonic probe 2.

The transmission controller 341 of the controller 34 delays a timing at which a drive voltage is applied to each ultrasonic transducer 45 (transmission and reception train Ch) so that ultrasonic waves output from the respective ultrasonic transducers 45 of the main transmission and reception portion 22A, the first sub-transmission and reception portion 22B, and the second sub-transmission and reception portion 22C pass through the reference plane P which is orthogonal to the ultrasonic wave transmission and reception direction D1 at the same time.

Consequently, ultrasonic waves passing through the reference plane P can be aligned with each other, and thus the ultrasonic waves are transmitted from the acoustic lenses 24A, 24B and 24C at the same time. In other words, wavefronts of transmitted ultrasonic waves can be aligned with each other, and thus ultrasonic waves having high sound pressure can be sent in the ultrasonic wave transmission and reception direction D1.

The reception controller 342 performs a reception process by delaying a reception timing of a received ultrasonic wave received by each ultrasonic transducer 45 (transmission and reception train Ch) by the delay time t corresponding to a position of the ultrasonic transducer 45. Consequently, it is possible to increase measurement accuracy in ultrasonic measurement, and thus for the image forming portion 343 to form an accurate internal tomographic image of a living body.

### Second Embodiment

Next, a second embodiment will be described.

In the first embodiment, a description has been made of an example in which the transmission controller 341 of the controller 34 performs delay control so that ultrasonic waves transmitted from the ultrasonic transducers 45 pass through the reference plane P at the same time. In contrast, the second embodiment is different from the first embodiment in that delay control is performed in the circuit substrates 60A, 60B and 60C of the ultrasonic probe 2.

An ultrasonic measurement apparatus 1 of the present embodiment has the substantially same configuration as in the first embodiment, and thus will be described with reference to the drawings used in the first embodiment.

In the present embodiment, the circuit substrate 60A provided on the main device 2A has the same configuration as in the first embodiment.

On the other hand, in the circuit substrate 60B provided in the first sub-device 2B, if a transmission command for transmitting an ultrasonic wave is input from the control device 3, the transmission circuit 62 applies a delayed drive voltage to each ultrasonic transducer 45 (transmission and reception train Ch) of the first sub-transmission and reception portion 22B. In other words, in the present embodiment, the transmission circuit 62 functions as a timing controller.

Since an acoustic distance of an ultrasonic wave is not variable in each of the sound guide portions 23A, 23B and 23C, a distance over which an ultrasonic wave output from each ultrasonic transducer 45 reaches the reference plane P is not variable either. Therefore, in the present embodiment, a delay time t until a drive voltage is applied to each ultrasonic transducer 45 (transmission and reception train Ch) of the sub-transmission and reception portions 22B and 22C after an ultrasonic wave transmission command is input to the main transmission and reception portion 22A is calculated in advance, and thus the transmission circuit 62 delays a timing of applying a drive voltage on the basis of the delay time t.

This is also the same for the reception circuit 63, and the reception circuit 63 of the present embodiment outputs a received signal to the control device 3 at a timing obtained by adding the delay time t calculated in advance to a signal output timing of the received signal output from each ultrasonic transducer 45.

In other words, in the present embodiment, the transmission circuit 62 and the reception circuit 63 of each of the circuit substrates 60B and 60C provided in the ultrasonic probe 2 function as a timing controller.

In the present embodiment, the transmission controller 341 of the control device 3 is not required to output a transmission command including the delay time t to the ultrasonic probe 2, and the reception controller 342 is not required to add the delay time t to a received signal which is input from the ultrasonic probe 2. Therefore, it is possible to simplify a process in the controller 34.

### Third Embodiment

Next, a third embodiment will be described.

In the first embodiment, the first sub-transmission and reception portion 22B or the second sub-transmission and reception portion 22C is inclined with respect to the main transmission and reception portion 22A. In contrast, the third embodiment is different from the first embodiment in that the first sub-transmission and reception portion 22B or the second sub-transmission and reception portion 22C is parallel to the main transmission and reception portion 22A.

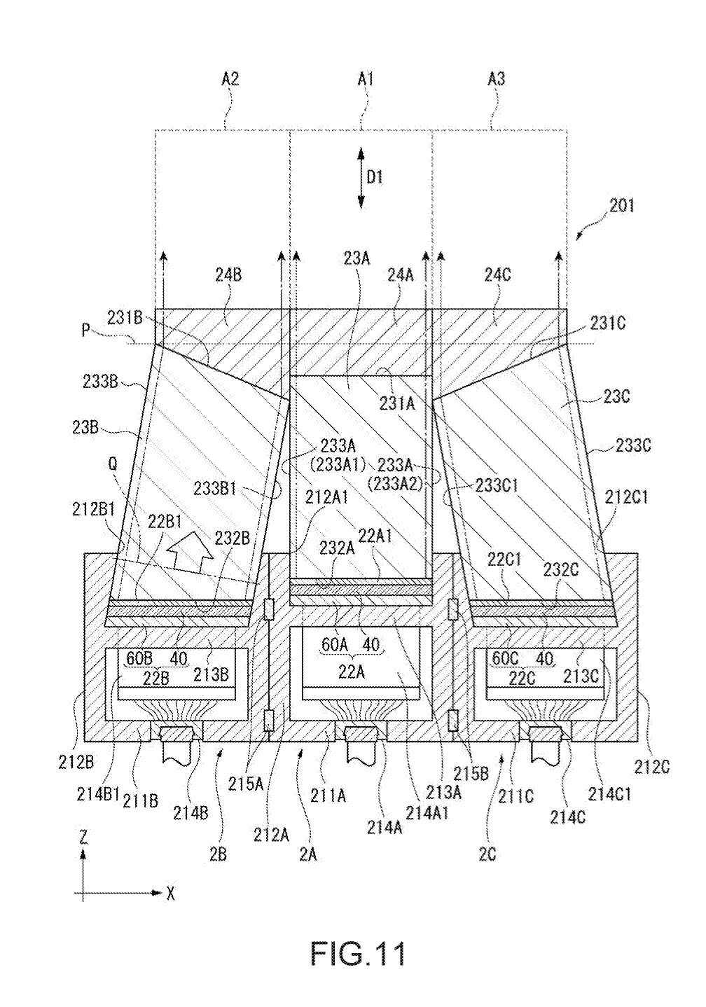

FIG. 11 is a schematic sectional view obtained by cutting an ultrasonic probe 201 in an XZ plane according to the third embodiment.

As illustrated in FIG. 11, in the present embodiment, the main transmission and reception portion 22A, the first sub-transmission and reception portion 22B, and the second sub-transmission and reception portion 22C are disposed in parallel to each other.

In the ultrasonic probe 201, as illustrated in FIG. 11, the transmission controller 341 of the control device 3 delays a drive timing of each ultrasonic transducer 45 so that an advancing direction of a wavefront (a two-dot chain line Q in FIG. 11) formed by combining ultrasonic waves transmitted from the first sub-transmission and reception portion 22B is directed toward the main sound guide portion 23A side (toward the first sub-distal end surface 231B). In other words, the ultrasonic transducers 45 are driven in a delay manner so that a drive timing of the ultrasonic transducer 45 disposed on the +X side is later than a drive timing of the ultrasonic transducer 45 disposed on the −X side.

In this case, more preferably, the transmission controller 341 delays a drive timing of each ultrasonic transducer 45 so that a wavefront formed by combining ultrasonic waves transmitted from the first sub-transmission and reception portion 22B with each other is perpendicular to the first sub-facing side surface 233B1 (an advancing direction of a combined wavefront is parallel to the first sub-facing side surface 233B1).

An inclined angle of the first sub-distal end surface 231B of the first sub-sound guide portion 23B is formed at an angle at which an advancing direction of a combined wavefront of ultrasonic waves transmitted from the first sub-transmission and reception portion 22B is refracted in the ultrasonic wave transmission and reception direction D1. In the same manner as in the first embodiment, the angle is set according to a difference or the like between sonic speeds in the first sub-acoustic lens 24B and the first sub-sound guide portion 23B.

The transmission controller 341 performs delay control on a drive timing of the first sub-transmission and reception portion 22B relative to a drive timing of the main transmission and reception portion 22A so that a wavefront of ultrasonic waves transmitted from the main transmission and reception portion 22A and a wavefront of ultrasonic waves transmitted from the first sub-transmission and reception portion 22B pass through the reference plane P at the same time.

Consequently, in the same manner as in the first embodiment, the ultrasonic wave transmission and reception range A2 in the first sub-transmission and reception portion 22B can be made close to the ultrasonic wave transmission and reception range A1 in the main transmission and reception portion 22A, and thus it is possible to perform uniform ultrasonic wave transmission and reception processes.

This is also the same for a drive method for the second sub-transmission and reception portion 22C, and for the second sub-distal end surface 231C.

An inclined angle of the second sub-distal end surface 231C of the second sub-sound guide portion 23C is formed at an angle at which an advancing direction of a combined wavefront of ultrasonic waves transmitted from the second sub-transmission and reception portion 22C is refracted in the ultrasonic wave transmission and reception direction D1.

The transmission controller 341 of the control device 3 delays a drive timing of each ultrasonic transducer 45 so that an advancing direction of a wavefront formed by combining ultrasonic waves transmitted from the second sub-transmission and reception portion 22C with each other is directed toward the main sound guide portion 23A side, and is more preferably parallel to the second sub-facing side surface 233C1. In addition thereto, the transmission controller 341 performs delay control on a drive timing of the second sub-transmission and reception portion 22C relative to a drive timing of the main transmission and reception portion 22A so that a wavefront of ultrasonic waves transmitted from the main transmission and reception portion 22A and a wavefront of ultrasonic waves transmitted from the second sub-transmission and reception portion 22C pass through the reference plane P at the same time.

Consequently, the ultrasonic wave transmission and reception range A3 in the second sub-transmission and reception portion 22C can be made close to the ultrasonic wave transmission and reception range A1 in the main transmission and reception portion 22A, and thus it is possible to perform uniform ultrasonic wave transmission and reception processes.

In the present embodiment, a description has been made of an example in which each ultrasonic transducer 45 is subject to delay driving under the control of the transmission controller 341 of the control device 3, but this is only an example, and, in the same manner as in the second embodiment, the above-described delay driving may be performed by the circuit substrates 60B and 60C provided in the ultrasonic probe 201.

In the present embodiment, the main transmission and reception portion 22A, the first sub-transmission and reception portion 22B, and the second sub-transmission and reception portion 22C are disposed in parallel to each other.

Even in a configuration in which the main transmission and reception portion 22A, the first sub-transmission and reception portion 22B, and the second sub-transmission and reception portion 22C are disposed in parallel to each other as in the present embodiment, as described above, it is possible to transmit and receive uniform ultrasonic waves with respect to a living body which is a subject. In other words, a plane on which the ultrasonic substrate 40 is disposed may be set as appropriate according to an arrangement relationship among other members of the devices 2A, 2B and 2C. Therefore, as in the first embodiment, the first sub-transmission and reception portion 22B and the second sub-transmission and reception portion 22C may be disposed to be inclined with respect to the main transmission and reception portion 22A, and, as in the present embodiment, the main transmission and reception portion 22A, the first sub-transmission and reception portion 22B, and the second sub-transmission and reception portion 22C may be disposed in parallel to each other. Thus, it is possible to improve a degree of freedom of design of each of the devices 2A, 2B and 2C.

### Fourth Embodiment

Next, a fourth embodiment will be described.

In the third embodiment, a description has been made of an example of performing delay driving on each ultrasonic transducer 45 of the sub-transmission and reception portions 22B and 22C so that an advancing direction of an ultrasonic wave is inclined toward the main sound guide portion 23A side. However, in the configuration, when ultrasonic waves are received, the ultrasonic waves are obliquely incident to the sub-transmission and reception portions 22B and 22C, and thus reception sensitivity is reduced. The fourth embodiment is different from the third embodiment in that a first sub-sound guide portion and a second sub-sound guide portion are formed of a plurality of layers, and thus a reduction in the reception sensitivity is suppressed even in a case where the main transmission and reception portion 22A, the first sub-transmission and reception portion 22B, and the second sub-transmission and reception portion 22C are disposed in parallel to each other.

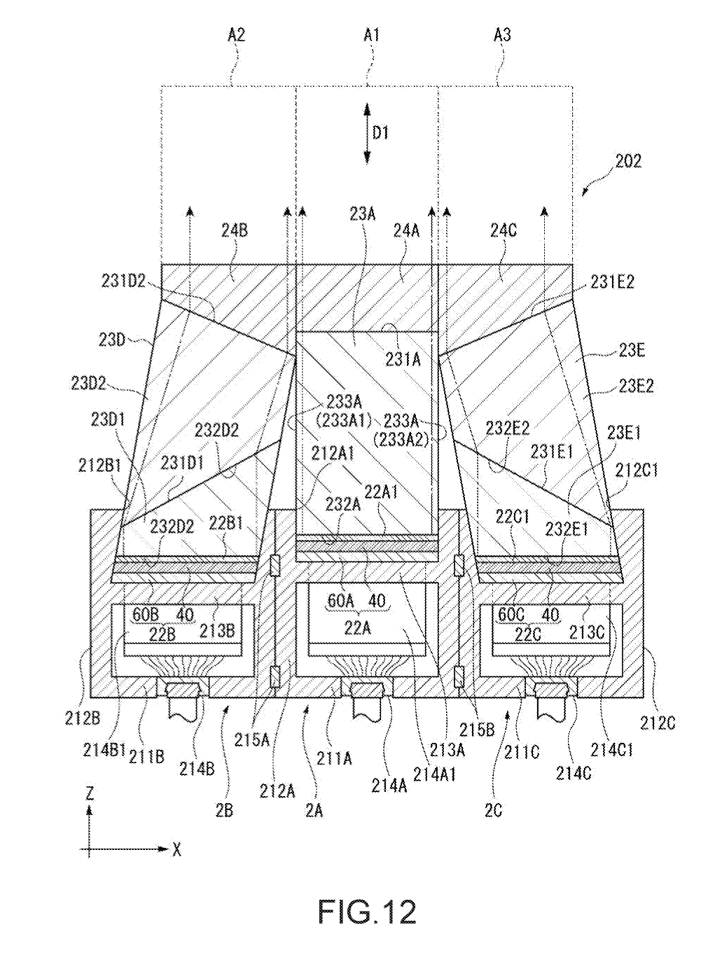

FIG. 12 is a schematic sectional view obtained by cutting an ultrasonic probe 202 in an XZ plane according to the fourth embodiment.

In the present embodiment, a first sub-sound guide portion 23D and a second sub-sound guide portion 23E are formed of a plurality of sound guide layers.

Specifically, as illustrated in FIG. 12, the first sub-sound guide portion 23D includes a first element side sound guide layer 23D1 disposed on the first sub-transmission and reception portion 22B side and a first lens side sound guide layer 23D2 disposed on the first sub-acoustic lens 24B side.

In the first element side sound guide layer 23D1, a surface (first element side distal end surface 231D1) on an opposite side to the first sub-transmission and reception portion 22B is an inclined surface whose distance from the first sub-transmission and reception portion 22B is increased in the +X direction.

On the other hand, a surface (first element side basal end surface 232D1; corresponding to a coupling surface) of the first element side sound guide layer 23D1 on the first sub-transmission and reception portion 22B side is fixed to the first sub-transmission and reception portion 22B via the acoustic layer 22B1.

In the first lens side sound guide layer 23D2, a surface (first lens side distal end surface 231D2; corresponding to a second distal end surface) on an opposite side to the first element side sound guide layer 23D1 is an inclined surface whose distance from the first sub-transmission and reception portion 22B is reduced in the +X direction.

On the other hand, a surface (first lens side basal end surface 232D2) of the first lens side sound guide layer 23D2 on the first element side sound guide layer 23D1 side is in close contact with and fixed to the first element side distal end surface 231D1.

The first sub-acoustic lens 24B, the first element side sound guide layer 23D1, and the first lens side sound guide layer 23D2 have different acoustic impedances. Specifically, the acoustic impedances are lowered in an order of the first element side sound guide layer 23D1, the first lens side sound guide layer 23D2, and the first sub-acoustic lens 24B. An inclined angle of the first element side distal end surface 231D1 and an inclined angle of the first lens side distal end surface 231D2 are respectively set so that an ultrasonic wave output from the ultrasonic transducer 45 located at a +X side end of the first sub-transmission and reception portion 22B is transmitted in the ultrasonic wave transmission and reception direction D1 from the +X side of the first lens side distal end surface 231D2 as illustrated in FIG. 12. The inclined angles are set as appropriate in the same manner as in the first embodiment according to a difference or the like between sonic speeds in the first sub-acoustic lens 24B, the first element side sound guide layer 23D1, and the first lens side sound guide layer 23D2.

The second sub-sound guide portion 23E also includes a second element side sound guide layer 23E1 disposed on the second sub-transmission and reception portion 22C side and a second lens side sound guide layer 23E2 disposed on the second sub-acoustic lens 24C side.

A second element side distal end surface 231E1 of the second element side sound guide layer 23E1 on an opposite side to the second sub-transmission and reception portion 22C is an inclined surface whose distance from the second sub-transmission and reception portion 22C is increased along the −X direction. A second element side basal end surface 232E1 of the second element side sound guide layer 23E1 on the second sub-transmission and reception portion 22C side is fixed to the second sub-transmission and reception portion 22C via the acoustic layer 22C1.

In the second lens side sound guide layer 23E2, a second lens side distal end surface 231E2 on an opposite side to the second element side sound guide layer 23E1 is an inclined surface whose distance from the second sub-transmission and reception portion 22C is reduced along the −X direction. A second lens side basal end surface 232E2 of the second lens side sound guide layer 23E2 on the second element side sound guide layer 23E1 side is in close contact with and fixed to the second element side distal end surface 231E1.

The second sub-acoustic lens 24C, the second element side sound guide layer 23E1, and the second lens side sound guide layer 23E2 have different acoustic impedances, and the acoustic impedances are lowered in an order of the second sub-acoustic lens 24C, the second element side sound guide layer 23E1, and the second lens side sound guide layer 23E2. An inclined angle of the second element side distal end surface 231E1 and an inclined angle of the second lens side distal end surface 231E2 are respectively set so that an ultrasonic wave output from the ultrasonic transducer 45 located at a −X side end of the second sub-transmission and reception portion 22C is transmitted in the ultrasonic wave transmission and reception direction D1 from the −X side of the second lens side distal end surface 231E2 as illustrated in FIG. 12.

Also in the present embodiment, in the same manner as in the first embodiment and the second embodiment, the controller 34 or the transmission circuit 62 performs delay control so that ultrasonic waves pass through the reference plane P at the same time when the ultrasonic waves are transmitted from the respective ultrasonic transducers 45 (transmission and reception train Ch).

The present embodiment achieves the same advantageous effects as in the third embodiment. In addition thereto, in the present embodiment, when an ultrasonic wave is received, the ultrasonic wave is incident to the first sub-transmission and reception portion 22B or the second sub-transmission and reception portion 22C from the normal direction. Thus, displacement of the vibration portion 421 of each ultrasonic transducer 45 can be increased, and thus a level of a received signal can be increased. Consequently, it is possible to improve reception sensitivity.

### Modification Examples

The invention is not limited to the above-described respective embodiments, and configurations obtained through modifications, alterations, and combinations of the embodiments within the scope of being capable of achieving the object of the invention are included in the invention.

### Modification Example 1

In the first embodiment, a description has been made of an exemplary configuration in which the first sub-device 2B, the main device 2A, and the second sub-device 2C are disposed along the X direction (scanning direction) in the ultrasonic probe 2, but any other configuration may be used.

For example, there may be a configuration in which a plurality of devices may be provided along the Y direction (slice direction).

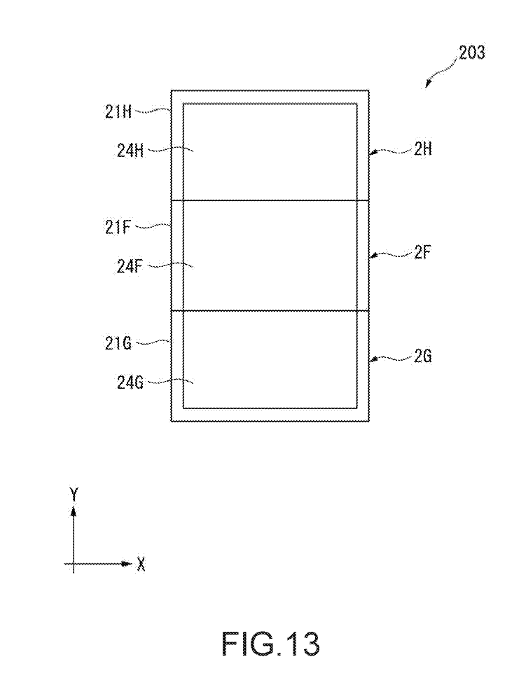

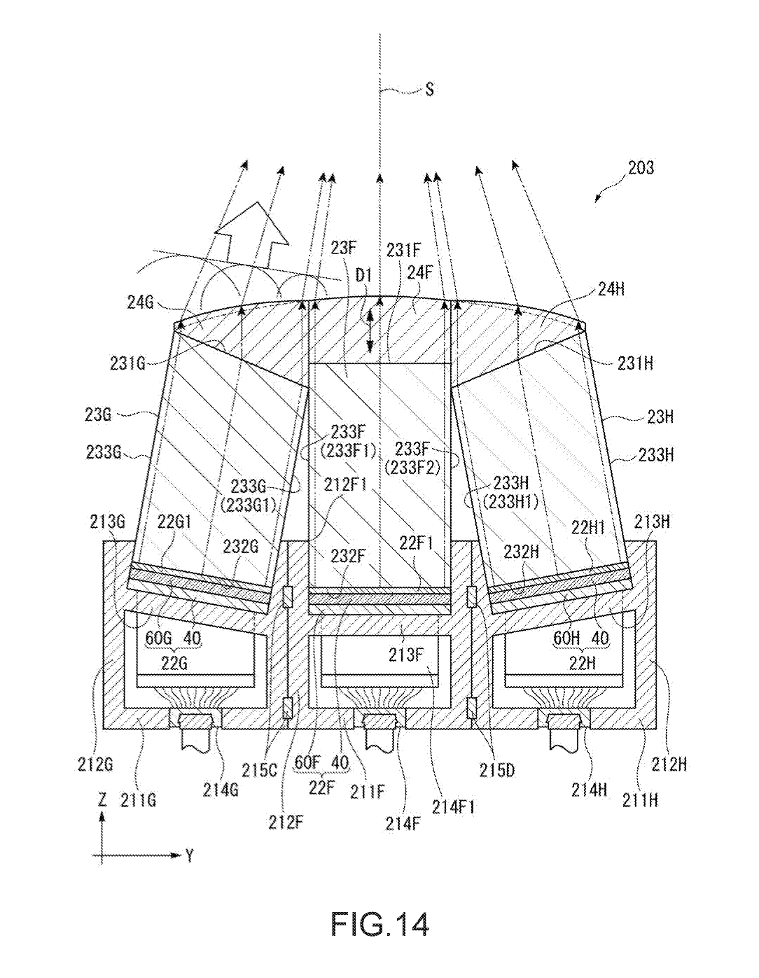

FIG. 13 is a plan view in which an ultrasonic probe 203 in Modification Example 1 is viewed from an ultrasonic wave transmission and reception direction. FIG. 14 is a sectional view illustrating a schematic configuration obtained by cutting the ultrasonic probe 203 in an YZ plane.

The ultrasonic probe 203 illustrated in FIG. 13 includes a main device 2F; a first sub-device 2G which is attachable to and detachable from a −Y side of the main device 2F; and a second sub-device 2H which is attachable to and detachable from a +Y side of the main device 2F.

In the same manner as in the first embodiment, the devices 2F, 2G and 2H respectively include casings 21F, 21G and 21H, and the casings 21F, 21G and 21H are respectively configured to include bottom portions 211F, 211G and 211H, sidewall portions 212F, 212G and 212H, pedestal portions 213F, 213G and 213H, and connectors 214F, 214G and 214H. The main casing 21F and the first sub-casing 21G are attachably and detachably fixed to each other via fixation mechanisms 215C, and the main casing 21F and the second sub-casing 21H are attachably and detachably fixed to each other via fixation mechanisms 215D.

A main transmission and reception portion 22F including an ultrasonic substrate 40 and a circuit substrate 60F are fixed to the pedestal portion 213F of the main device 2F; a first sub-transmission and reception portion 22G including an ultrasonic substrate 40 and a circuit substrate 60G is fixed to the pedestal portion 213G of the first sub-device 2G; and a second sub-transmission and reception portion 22H including an ultrasonic substrate 40 and a circuit substrate 60H is fixed to the pedestal portion 213H of the second sub-device 2H.

In the present example, in the same manner as in the first embodiment, the pedestal portion 213G and the pedestal portion 213H are inclined toward the main device 2F side. In the present example, for simplification of description, a description will be made of an example in which the ultrasonic transducer 45 of the first sub-transmission and reception portion 22G on the +Y side end and the ultrasonic transducer 45 of the second sub-transmission and reception portion 22H on the −Y side end are located at the same height as that of the ultrasonic transducer 45 of the main transmission and reception portion 22F in the ultrasonic wave transmission and reception direction D1 (Z direction).

A main sound guide portion 23F and a main acoustic lens 24F are provided on the main transmission and reception portion 22F via an acoustic layer 22F1. A first sub-sound guide portion 23G and a first sub-acoustic lens 24G are provided on the first sub-transmission and reception portion 22G via an acoustic layer 22G1, and a second sub-sound guide portion 23H and a second sub-acoustic lens 24H are provided on the second sub-transmission and reception portion 22H via an acoustic layer 22H1.

The main sound guide portion 23F, the first sub-sound guide portion 23G, and the second sub-sound guide portion 23H have the same configurations as those of the sound guide portions 23A, 23B and 23C in the first embodiment. In other words, the main sound guide portion 23F causes an ultrasonic wave transmitted and received in the main transmission and reception portion 22F to propagate between a main distal end surface 231F and a main basal end surface 232F. The first sub-sound guide portion 23G causes an ultrasonic wave transmitted and received in the first sub-transmission and reception portion 22G to propagate between a first sub-distal end surface 231G and a first sub-basal end surface 232G. The second sub-sound guide portion 23H causes an ultrasonic wave transmitted and received in the second sub-transmission and reception portion 22H to propagate between a second sub-distal end surface 231H and a second sub-basal end surface 232H.

The first sub-sound guide portion 23G and the second sub-sound guide portion 23H are inclined toward the main sound guide portion 23F side, a +Y side end of the first sub-distal end surface 231G is in contact with the main sound guide portion 23F, and a −Y side end of the second sub-distal end surface 231H is in contact with the main sound guide portion 23F. An air layer is interposed between a main side surface 233F (first main side surface 233F1) of the main sound guide portion 23F and a first sub-side surface 233G (first sub-facing side surface 233G1), and an air layer is interposed between the main side surface 233F (second main side surface 233F2) of the main sound guide portion 23F and a second sub-side surface 233H (second sub-facing side surface 233H1).

In Modification Example 1, the main acoustic lens 24F is formed in a cylindrical shape, and a surface on a living body side has a circular arc shape, in the same manner as in the first embodiment.

In the first sub-acoustic lens 24G, an end of the surface on the living body side on the main acoustic lens 24F is connected to the −Y side end of the main acoustic lens 24F. A surface of the first sub-acoustic lens 24G on the living body side has a circular arc shape which comes closer to the first sub-sound guide portion 23G toward the end on the −Y side from the end on the main acoustic lens 24F side.

Also in the second sub-acoustic lens 24H, an end of the surface on the living body side on the main acoustic lens 24F is connected to the +Y side end of the main acoustic lens 24F. A surface of the second sub-acoustic lens 24H on the living body side has a circular arc shape which comes closer to the second sub-sound guide portion 23H toward the end on the +Y side from the end on the main acoustic lens 24F side.

In the ultrasonic probe 203, the ultrasonic transducers 45 arranged in the Y direction are simultaneously driven in each ultrasonic substrate 40. Thus, timings at which ultrasonic waves are emitted from the acoustic lenses 24G and 24H are different from timings depending on positions.

For example, in the first sub-device 2G, a timing at which an ultrasonic wave transmitted from the −Y side of the first sub-transmission and reception portion 22G is transmitted from the first sub-acoustic lens 24G is earlier than a timing at which an ultrasonic wave transmitted from the +Y side of the first sub-transmission and reception portion 22G is transmitted from the first sub-acoustic lens 24G. Therefore, in a case where a wavefront is obtained by combining the ultrasonic waves with each other, the ultrasonic wave advances toward a +Y+Z side (toward a scan surface S including the central axis in the Y direction, and the Z direction). In addition thereto, an advancing direction of the ultrasonic wave is further modified when the ultrasonic wave passes through the circular arc surface of the first sub-acoustic lens 24G, and the ultrasonic wave is transmitted so as to converge at a predetermined position on the scan surface S. This is also the same for the second sub-device 2H, and a wavefront obtained by combining ultrasonic waves from the acoustic lens 24H with each other advances so as to converge at a predetermined position on the scan surface S.

On the other hand, in the main device 2F, a distal end of the main acoustic lens 24F has a circular arc shape in the same manner as in the first embodiment. Therefore, an ultrasonic wave emitted from the main transmission and reception portion 22F is transmitted so as to converge at a predetermined position on the scan surface S.

Consequently, also in the ultrasonic probe 203 of Modification Example 1, in the same manner as in the first embodiment, it is possible to output an ultrasonic wave which converges at a predetermined position on the scan surface with respect to the slice direction.

As illustrated in FIG. 14, each of curvatures of the circular arcs of the surface of the first sub-acoustic lens 24G on a living body side and the surface of the second sub-acoustic lens 24H on the living body side may be smaller than a curvature of the surface of the main acoustic lens 24F on the living body side. A dashed line in FIG. 14 indicates the surfaces of the first sub-acoustic lens 24B and the second sub-acoustic lens 24C on the living body side of the first embodiment. In the present modification example, since ultrasonic waves are simultaneously transmitted from the respective ultrasonic transducers 45 in the slice direction (X direction), an ultrasonic wave sent from the +X side (main sound guide portion 23F side) of the first sub-sound guide portion 23G is delayed rather than an ultrasonic wave sent from the −X side thereof. As illustrated in FIG. 14, a curvature of the circular arc of the surface of the first sub-acoustic lens 24G on the living body side is made smaller than a curvature of that of the main acoustic lens 24F, and thus it is possible to reduce the influence of the delay. This is also the same for the second sub-acoustic lens 24H.

### Modification Example 2

In the above Modification Example 1, each ultrasonic substrate 40 has a one-dimensional array structure in which the ultrasonic transducers 45 arranged in the Y direction are simultaneously driven, but the ultrasonic transducers 45 of each ultrasonic substrate 40 may be configured in a two-dimensional array structure. In this case, delay driving is performed on each ultrasonic transducer 45, and thus ultrasonic waves output from the respective ultrasonic transducers 45 can be output so as to converge at a predetermined position on the scan surface S.

### Modification Example 3

In Modification Example 1, a description has been made of an example in which the first sub-transmission and reception portion 22G or the second sub-transmission and reception portion 22H is inclined with respect to the main transmission and reception portion 22F. In contrast, in the same manner as in the third embodiment, the first sub-transmission and reception portion 22G or the second sub-transmission and reception portion 22H may be provided in parallel to the main transmission and reception portion 22F.

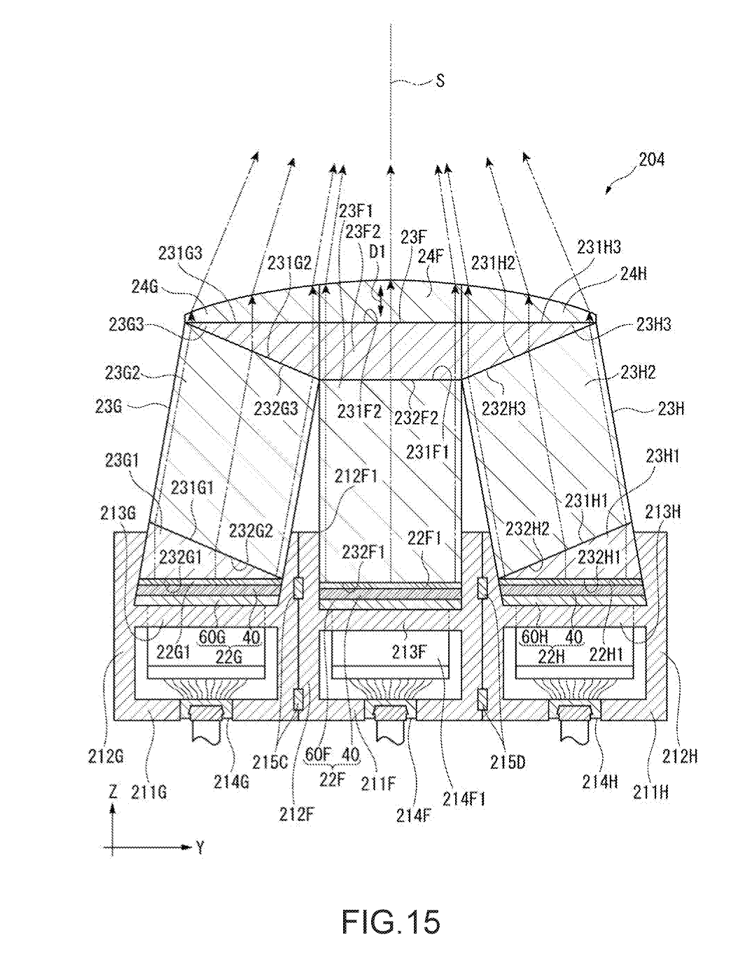

FIG. 15 is a schematic sectional view obtained by cutting an ultrasonic probe 204 in a YZ plane according to Modification Example 3.

In the ultrasonic probe 204, the main transmission and reception portion 22F, the first sub-transmission and reception portion 22G, and the second sub-transmission and reception portion 22H are provided on the same plane.

The first sub-sound guide portion 23G includes a first element side sound guide part 23G1, a first intermediate sound guide part 23G2, and a first lens side sound guide part 23G3.

The first element side sound guide part 23G1 and the first lens side sound guide part 23G3 have the same acoustic impedance, and the first intermediate sound guide part 23G2 has an acoustic impedance higher than that of the first element side sound guide part 23G1.

A distal end surface (first element side distal end surface 231G1) of the first element side sound guide part 23G1 and a basal end surface (first lens side basal end surface 232G3) of the first lens side sound guide part 23G3 are parallel to each other, and are inclined with respect to the ultrasonic wave transmission and reception direction D1. A basal end surface (second intermediate basal end surface 232G2) of the first intermediate sound guide part 23G2 is in contact with the first element side distal end surface 231G1, and a distal end surface (second intermediate distal end surface 231G2) of the first intermediate sound guide part 23G2 is in contact with the first lens side basal end surface 232G3. In other words, a thickness dimension of the first intermediate sound guide part 23G2 is uniform.

The first sub-transmission and reception portion 22G is fixed to a basal end surface (first element side basal end surface 232G1) of the first element side sound guide part 23G1 via the acoustic layer 22G1. A distal end surface (first lens side distal end surface 231G3) of the first lens side sound guide part 23G3 is parallel to the first element side basal end surface 232G1.

Similarly, the second sub-sound guide portion 23H includes a second element side sound guide part 23H1, a second intermediate sound guide part 23H2, and a second lens side sound guide part 23H3.

The second element side sound guide part 23H1 and the second lens side sound guide part 23H3 have the same acoustic impedance, and the second intermediate sound guide part 23H2 has an acoustic impedance higher than that of the second element side sound guide part 23H1.

A distal end surface (second element side distal end surface 231H1) of the second element side sound guide part 23H1 and a basal end surface (second lens side basal end surface 232H3) of the second lens side sound guide part 23H3 are parallel to each other, and are inclined with respect to the ultrasonic wave transmission and reception direction D1. A basal end surface (second intermediate basal end surface 232H2) of the second intermediate sound guide part 23H2 is in contact with the second element side distal end surface 231H1, and a distal end surface (second intermediate distal end surface 231H2) of the second intermediate sound guide part 23H2 is in contact with the second lens side basal end surface 232H3.

The second sub-transmission and reception portion 22H is fixed to a basal end surface (second element side basal end surface 232H1) of the second element side sound guide part 23H1 via the acoustic layer 22H1. A distal end surface (second lens side distal end surface 231H3) of the second lens side sound guide part 23H3 is parallel to the second element side basal end surface 232H1.

The main sound guide portion 23F includes a main element side sound guide part 23F1 and a main lens side sound guide part 23F2.

The main element side sound guide part 23F1 is made of the same material as that of the first intermediate sound guide part 23G2 or the second intermediate sound guide part 23H2, and thus has the same acoustic impedance. The main transmission and reception portion 22F is fixed to a basal end surface (main element side basal end surface 232F1) of the main element side sound guide part 23F1 via the acoustic layer 22F1. A dimension from a distal end surface (main element side distal end surface 231F1) of the main element side sound guide part 23F1 to the main element side basal end surface 232F1, that is, a thickness dimension of the main element side sound guide part 23F1 is the same as a thickness dimension of the first intermediate sound guide part 23G2 or the second intermediate sound guide part 23H2.

The main lens side sound guide part 23F2 is made of the same material as that of the first element side sound guide part 23G1, the first lens side sound guide part 23G3, the second element side sound guide part 23H1, and the second lens side sound guide part 23H3, and has the same acoustic impedance. A basal end surface (main lens side basal end surface 232F2) of the main lens side sound guide part 23F2 is in contact with the main element side distal end surface 231F1.

A side surface of the main lens side sound guide part 23F2 on the −Y side is in contact with a side surface of the first lens side sound guide part 23G3, and a side surface of the main lens side sound guide part 23F2 on the +Y side is in contact with a side surface of the second lens side sound guide part 23H3.

A dimension from a distal end surface (main lens side distal end surface 231F2) of the main lens side sound guide part 23F2 to the main lens side basal end surface 232F2, that is, a thickness dimension of the main lens side sound guide part 23F2 is the same as a dimension of a side surface (a side surface of the second lens side sound guide part 23H3 on the −Y side) of the first lens side sound guide part 23G3 on the +Y side in the Z direction (ultrasonic wave transmission and reception direction D1).

As illustrated in FIG. 15, in the ultrasonic probe 204, acoustic distances over which ultrasonic waves output from each ultrasonic transducers 45 of the respective ultrasonic wave transmission and reception portions 22F, 22G and 22H reach the reference plane P are the same as each other. Therefore, an ultrasonic wave emission surface formed by the main acoustic lens 24F, the first sub-acoustic lens 24G, and the second sub-acoustic lens 24H may be formed in a circular arc shape in the same manner as in the first embodiment.

In the present modification example, delay control is not required to be performed on an ultrasonic wave, and thus it is possible to simplify ultrasonic wave transmission and reception processes.

In the present modification example, a description has been made of an example in which the respective devices 2F, 2G and 2H are arranged in the Y direction (scanning direction), but this is only an example. Instead of the sub-sound guide portions 23D and 23E of the fourth embodiment, as described above, a configuration including the intermediate sound guide parts having the uniform thickness dimension may be used.

### Modification Example 4

In the first to fourth embodiments, a description has been made of an exemplary configuration in which a plurality of ultrasonic wave transmission and reception portions, sound guide portions, and acoustic lenses are disposed in the X direction (scanning direction), and, in Modification Examples 1 to 3, a description has been made of an exemplary configuration in which a plurality of ultrasonic wave transmission and reception portions, sound guide portions, and acoustic lenses are disposed in the Y direction (slice direction), but any other configuration may be used.

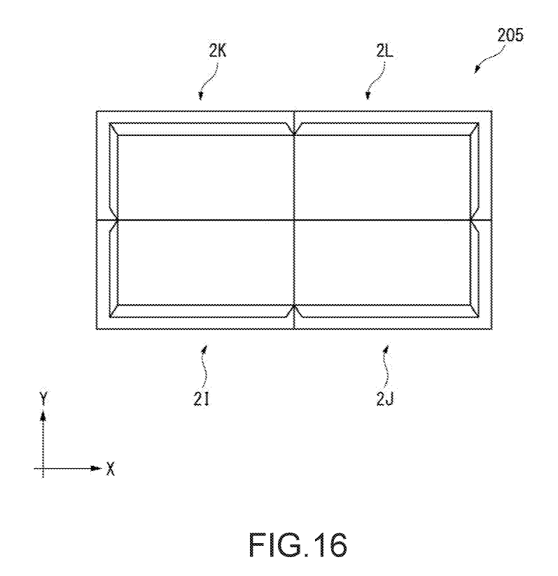

FIG. 16 is a plan view in which an ultrasonic probe 205 in Modification Example 4 is viewed from an ultrasonic wave transmission and reception direction.

As illustrated in FIG. 16, respective devices 2I, 2J, 2K and 2L may be disposed in the X direction and the Y direction.

### Modification Example 5

In the respective embodiments and modification examples, a description has been made of the ultrasonic wave transmission and reception direction D1 which is parallel to the Z direction as an example, and this is only an example. As a direction in which an ultrasonic wave is transmitted from an ultrasonic probe, for example, a direction inclined at a predetermined angle with respect to the Z direction may be an ultrasonic wave transmission and reception direction. In this case, when viewed from the ultrasonic wave transmission and reception direction, a first distal end surface (main distal end surface 231A or 231F) of a first sound guide portion (main sound guide portion 23A or 23F) is made close (preferably adjacent) to a second distal end surface (the first sub-distal end surface 231B or 231G, the first lens side distal end surface 231D2 or 231G3, the second sub-distal end surface 231C or 231H, or the second lens side distal end surface 231E2 or 231H3) of a second sound guide portion (the first sub-sound guide portion 23B, 23D or 23G, or the second sub-sound guide portion 23C, 23E or 23H), and thus it is possible to transmit and receive uniform ultrasonic waves.

A description has been made of an example in which the first distal end surface and the second distal end surface are adjacent to each other when viewed from the ultrasonic wave transmission and reception direction D1, but the first distal end surface and the second distal end surface may be separated from each other by a predetermined distance. Also in this case, if a distance between the first distal end surface of the first sound guide portion and the second distal end surface of the second sound guide portion is shorter than a distance between the first ultrasonic wave transmission and reception portion and the second ultrasonic wave transmission and reception portion, it is possible to transmit and receive uniform ultrasonic waves compared with a configuration in which the first sound guide portion or the second sound guide portion is not provided.

### Modification Example 6

In the respective embodiments and modification examples, a description has been made of an exemplary configuration in which the ultrasonic probe is formed of a plurality of devices, and the devices can be attached to and detached from each other, but any other configuration may be used. For example, there may be a configuration in which respective devices are fixed and are integrally formed.

In this case, there may be a configuration in which the main transmission and reception portion 22A (22F), the first sub-transmission and reception portion 22B (22G), and the second sub-transmission and reception portion 22C (22H) are provided on the same substrate. For example, an ultrasonic array forming the main transmission and reception portion 22A, an ultrasonic array forming the first sub-transmission and reception portion 22B, and an ultrasonic array forming the second sub-transmission and reception portion 22C may be provided on a single ultrasonic substrate, and the circuit substrates 60A, 60B and 60C controlling the ultrasonic substrate may be integrated into a single substrate.

Also in this case, the respective sound guide portions 23A, 23B and 23C may be integrally formed, and the acoustic lenses 24A, 24B and 24C may be integrally formed.

In each of the devices 2A, 2B and 2C, the sound guide portion and the acoustic lens (for example, the main sound guide portion 23A and the main acoustic lens 24A) may be integrally formed. Here, in a case where the first sub-sound guide portion 23B and the first sub-acoustic lens 24B are integrally formed, delay control may be performed on drive timings of the respective ultrasonic transducers 45 (transmission and reception train Ch) so that ultrasonic waves sent from the respective ultrasonic transducers 45 (transmission and reception train Ch) in the first sub-transmission and reception portion 22B pass through the reference plane P at the same time. This is also the same for the second sub-sound guide portion 23C and the second sub-acoustic lens 24C. There may be a configuration in which the sound guide portions 23A, 23B and 23C and the acoustic lenses 24A, 24B and 24C may also be integrally formed.

### Modification Example 7

In the first embodiment, a description has been made of an example in which one end of the first sub-distal end surface 231B or the second sub-distal end surface 231C is in contact with the side surface of the main sound guide portion 23A, but may be in contact with the end of the main distal end surface 231A. In other words, there may be a configuration in which the main distal end surface 231A, the first sub-distal end surface 231B, and the second sub-distal end surface 231C are continuous surfaces.

There may be a configuration in which a dimension of the main sound guide portion 23A in the Z direction is small, and the tip of the first sub-distal end surface 231B or the second sub-distal end surface 231C is in contact with the side surface of the main acoustic lens 24A.

As in the respective embodiments, in a case where an ultrasonic wave transmission and reception direction of the main transmission and reception portion 22A is a normal direction to the main transmission and reception portion 22A, the main sound guide portion 23A may be integrally formed with the main acoustic lens 24A. In other words, the main sound guide portion 23A and the main acoustic lens 24A may be made of the same material (the same acoustic impedance).

### Modification Example 8

In the respective embodiments and modification examples, a description has been made of an example in which the air layer is provided between the main sound guide portion and the sub-sound guide portion, but this is only an example.

For example, an intermediate portion having an acoustic impedance which is different from that of the sound guide portions may be provided between the main sound guide portion and the sub-sound guide portion.

Such an intermediate portion may be, for example, a part of a casing forming each device, and may be a fixation mechanism which attachably and detachably fixes devices to each other.

The intermediate portion may not be provided. In a case where the intermediate portion is not provided, there may be a configuration in which a single sound guide body into which the first sound guide portion and the second sound guide portion are integrated is provided. For example, there may be a configuration in which the main sound guide portion 23A is connected to the first sub-sound guide portion 23B via the same material as that of the sound guide portions.

### Modification Example 9

A description has been made of an example in which the sealing plate 50 is provided on the support film 42 side of the element base plate 41 as the ultrasonic transducer 45, an ultrasonic wave is transmitted from the opening portion 41A of the element base plate 41, and an ultrasonic wave incident from the opening portion 41A is received, but this is only an example. For example, there may be a configuration in which the sealing plate 50 is provided on an opposite side to the support film 42 of the element base plate 41, an ultrasonic wave is transmitted toward an opposite side to the opening portion 41A, and an ultrasonic wave incident from the opposite side to the opening portion 41A is received.

A description has been made of an example in which the lower electrode 431 is used as a drive signal line, but the upper electrode 433 may be used as a drive signal line.

In the above-described embodiments, a description has been made of an exemplary configuration in which the ultrasonic transducer 45 includes the support film 42, and the piezoelectric element 43 which causes the vibration film to vibrate as an ultrasonic element. However, this is only an example, and ultrasonic elements other than the piezoelectric element may be used. For example, there may be the use of an ultrasonic element in which a vibration film is disposed on a substrate via an air gap, and an electrostatic actuator is disposed between the substrate and the vibration film so as to cause the vibration film to vibrate.

The ultrasonic transducer 45 may be configured to cause a vibrator such as a piezoelectric element to vibrate so that an ultrasonic wave is transmitted, without including a vibration film.

### Modification Example 10

In the above-described embodiments, a description has been made of the ultrasonic measurement apparatus 1 in which a living body is used as a subject, but this is only an example. For example, the invention is applicable to an ultrasonic apparatus which employs various structural bodies as subjects, and detects defects of the structural bodies or examines deterioration thereof. For example, the invention is applicable to an ultrasonic apparatus which employs various semiconductor packages, wafers, or the like as subjects, and detects defects of the subjects.

A specific structure at the time of implementing the invention may be configured by combining the respective embodiments and modification examples with each other as appropriate within the scope of being capable of achieving the object of the invention, and may be altered to other structures as appropriate.

The entire disclosure of Japanese Patent Application No. 2016-220472 filed Nov. 11, 2016 is expressly incorporated by reference herein.

What is claimed is:

1. An ultrasonic unit comprising:
a first and a second transmission and reception portions that transmit ultrasonic waves to a subject and receive ultrasonic waves reflected from the subject;
a first sound guide portion through which ultrasonic waves transmitted and received by a first ultrasonic wave transmission and reception portion pass; and
a second sound guide portion through which ultrasonic waves transmitted and received by a second ultrasonic wave transmission and reception portion pass,
wherein a distance between the first sound guide portion and the second sound guide portion is gradually reduced toward the subject.

2. The ultrasonic unit according to claim 1, further comprising:
an intermediate portion that has an acoustic impedance which is different from an acoustic impedance of each of the first sound guide portion and the second sound guide portion is provided between the first sound guide portion and the second sound guide portion.

3. The ultrasonic unit according to claim 2,
wherein the intermediate portion includes an air layer.

4. The ultrasonic unit according to claim 1,
wherein facing side surfaces of the first sound guide portion and the second sound guide portion are inclined in intersecting directions toward the subject.

5. The ultrasonic unit according to claim 1,
wherein a coupling surface of the first ultrasonic wave transmission and reception portion and the first sound guide portion and a coupling surface of the second ultrasonic wave transmission and reception portion and the second sound guide portion are located on different planes.

6. The ultrasonic unit according to claim 1, further comprising:
a first device including the first ultrasonic wave transmission and reception portion and the first sound guide portion; and
a second device including the second ultrasonic wave transmission and reception portion and the second sound guide portion,
wherein the first device and the second device are attachable to and detachable from each other.

7. An ultrasonic apparatus comprising:
the ultrasonic unit according to claim 1; and
a controller that controls the ultrasonic unit.

8. An ultrasonic apparatus comprising:
the ultrasonic unit according to claim 2; and
a controller that controls the ultrasonic unit.

9. An ultrasonic apparatus comprising:
the ultrasonic unit according to claim 3; and
a controller that controls the ultrasonic unit.

10. An ultrasonic apparatus comprising:
the ultrasonic unit according to claim 4; and
a controller that controls the ultrasonic unit.

11. An ultrasonic apparatus comprising:
the ultrasonic unit according to claim 5; and
a controller that controls the ultrasonic unit.

12. An ultrasonic apparatus comprising:
the ultrasonic unit according to claim 6; and
a controller that controls the ultrasonic unit.

13. The ultrasonic apparatus according to claim 7,
wherein the controller includes a timing controller that controls transmission timings of ultrasonic waves so that ultrasonic waves transmitted from the plurality of ultrasonic wave transmission and reception portions are simultaneously incident to a reference plane intersecting a direction directed toward the subject.

14. A control method for the ultrasonic unit according to claim 1, the method comprising:
performing delay driving on the plurality of ultrasonic wave transmission and reception portions so that ultrasonic waves transmitted from the first and the second ultrasonic wave transmission and reception portions are simultaneously incident to a reference plane intersecting a direction directed toward the subject.

15. A control method for the ultrasonic unit according to claim 2, the method comprising:
performing delay driving on the plurality of ultrasonic wave transmission and reception portions so that ultrasonic waves transmitted from the first and the second ultrasonic wave transmission and reception portions are simultaneously incident to a reference plane intersecting a direction directed toward the subject.

16. A control method for the ultrasonic unit according to claim 3, the method comprising:
performing delay driving on the plurality of ultrasonic wave transmission and reception portions so that ultrasonic waves transmitted from the first and the second ultrasonic wave transmission and reception portions are simultaneously incident to a reference plane intersecting a direction directed toward the subject.

17. A control method for the ultrasonic unit according to claim 4, the method comprising:
performing delay driving on the plurality of ultrasonic wave transmission and reception portions so that ultrasonic waves transmitted from the first and the second ultrasonic wave transmission and reception portions are simultaneously incident to a reference plane intersecting a direction directed toward the subject.

18. A control method for the ultrasonic unit according to claim 5, the method comprising:
performing delay driving on the plurality of ultrasonic wave transmission and reception portions so that ultrasonic waves transmitted from the first and the second ultrasonic wave transmission and reception portions are simultaneously incident to a reference plane intersecting a direction directed toward the subject.

19. A control method for the ultrasonic unit according to claim 6, the method comprising:
performing delay driving on the plurality of ultrasonic wave transmission and reception portions so that ultrasonic waves transmitted from the first and the second ultrasonic wave transmission and reception portions are simultaneously incident to a reference plane intersecting a direction directed toward the subject.
